(19) United States
(12) Patent Application Publication
(10) Pub. No.: US 2023/0009415 A1
(43) Pub. Date: Jan. 12, 2023

(54) SYNTHETIC BIOMARKERS FOR DIFFERENTIAL SEROLOGICAL DIAGNOSIS OF CUTANEOUS LEISHMANIASIS (CL) CAUSED BY VARIOUS LEISHMANIA SPECIES

(71) Applicant: BOARD OF REGENTS, THE UNIVERSITY OF TEXAS SYSTEM, Austin, TX (US)

(72) Inventors: Katja Michael, El Paso, TX (US); Igor C. Almeida, El Paso, TX (US); Rosa A. Maldonado, El Paso, TX (US); Alvaro Acosta-Serrano, El Paso, TX (US); Alba A. Montoya, El Paso, TX (US); Susana Portillo, El Paso, TX (US)

(21) Appl. No.: 17/856,241

(22) Filed: Jul. 1, 2022

Related U.S. Application Data
(60) Provisional application No. 63/217,727, filed on Jul. 1, 2021.

(51) Int. Cl. G01N 33/569; A61P 33/02; A61K 47/64; A61K 47/42; A61K 47/65

(52) U.S. Cl. CPC G01N 33/569; A61P 33/02; A61K 47/64; A61K 47/42; A61K 47/65

(57) ABSTRACT

Disclosed are neoglycoconjugates and/or glycosides containing glycan selected from Galpα1,3Galfβ, Galpα1,6Galpα1,3Galfβ, or Galpα1,3Galfβ1,3Manpα. Methods of using the glycosides and/or neoglycoconjugates as diagnostic or prognostic biomarkers, vaccines, treating or detecting parasitic diseases, such as cutaneous leishmaniasis are disclosed.

### RELATED APPLICATION

This Application claims priority to U.S. Provisional Application 63/217,727 filed Jul. 1, 2021, which is incorporated herein by reference in its entirety.

### STATEMENT REGARDING FEDERALLY FUNDED RESEARCH

This invention was made with government support under 1R21AI137890-01 awarded by the National Institutes of Health. The government has certain rights in the invention.

### BACKGROUND

Old-World primates, including humans, do not express α-galactose (α-Gal) in their glycoproteins due to the inactivation of the α1,3-galactosyltransferase gene in ancestral Old-World primates 20-30 million years ago (Galili et al., J. Biol. Chem. 1988, 263, 17755-62). Therefore, humans can produce anti-α-galactosyl antibodies (anti-α-Gal Abs) against α-Gal-bearing antigens. Due to continuous stimulation by antigenic α-Gal-containing lipopolysaccharides of enterobacteria (Galili et al., Infect. Immun. 1988, 56, 1730-37), high titers of anti-α-Gal Abs are maintained life-long in normal human serum (NHS) (Galili et al., J. Exp. Med. 1984, 160, 1519-31). The exact structures of the glycotopes that collectively elicit these anti-α-Gal Abs are yet to be identified and structurally characterized. While it is unknown whether natural anti-α-Gal Abs have a distinct physiological purpose, they are important players in immunoglycomics due to their cross-reactivity with other α-Gal-containing glycans. For example, their cross-reactivity with the Galili trisaccharide, Galα1,3Galβ1,4GlcNAcβ, which is expressed by all mammals other than Old-World primates (Galili et al., J. Biol. Chem. 1988, 263, 17755-62), is responsible for the hyperacute rejection of porcine xenografts (Galili, Immunol. Today 1993, 14, 480-82), On the other hand, they are likely to interfere with the transmission of α-Gal-containing enveloped animal viruses to humans, thus protecting humans from zoonotic viral diseases (Galili et al., J. Biol. Chem. 1988, 263, 17755-62). The binding of natural anti-α-Gal Abs from NHS (or from α1,3-galactosyltransferase-knockout mice or pigs) to Galα1,3Galβ-containing glycans has been exploited in a number of biomedical contexts (Galili et al., J. Biol. Chem. 1988, 263, 17755-62). Examples include the anti-α-Gal Abs-mediated recruitment of macrophages by α-Gal nanoparticles in wound healing and tissue repair, the decoration of cancer cells and viral and bacterial pathogens with α-Gal-containing bifunctional molecules to increase their antigenicity and cause their destruction by the immune system (Carlson et al., ACS Chem. Biol. 2007, 2, 119-27; Sianturi et al., Angew. Chem. Int. Ed. Engl. 2019, 58, 4526-30; Galili, 2018) and the development of α-Gal-expressing tumor cells as anti-cancer vaccines (LaTemple et al., Cancer Res. 1999, 56, 3069-74). Furthermore, α-Gal residues of glycoengineered protein- and virus-based vaccines form immunocomplexes with anti-α-Gal Abs, serving as adjuvants leading to enhanced uptake by antigen-presenting cells (Galili, Vaccine 2020, 38, 6487-99). The natural (or NHS) anti-α-Gal Abs are also known to cross-react with α-Gal-expressing protozoa, i.e., some Leishmania spp. (Avila et al., J Immunol 1989, 142, 2828-34; Avila, Subcell Biochem 1999, 32, 173-213), Trypanosoma cruzi (Avila et al., J Immunol 1989, 142, 2828-34; Almeida et al., J Immunol 1991, 146, 2394-400; Milani and Travassos, Braz J Med Biol Res 1988, 21, 1275-86), Trypanosoma brucei, (Ramasamy and Field, Ep Parasitol 2012, 130, 314-20), and Plasmodium falciparum (Aguilar et al., Sci Rep 2018, 8, 9999; Ravindran et al., Immunol Lett 1988, 19, 137-41; Ramasamy and Reese, Mol Biochem Parasitol 1986, 19, 91-101), the causative agents of leishmaniasis, Chagas disease (CD), African trypanosomiasis, and malaria, respectively. These antibodies could be the first line of defense to fend off the establishment of parasitic infections in Old-World primates (Galili, 2018). In patients with these infectious diseases, parasitic α-Gal-bearing glycotopes elicit specific anti-α-Gal Abs that exceed the NHS anti-α-Gal Abs in both concentration and binding strength and, ultimately, in specificity (Avila et al., J Immunol 1989, 142, 2828-34; Almeida et al., J Immunol 1991, 146, 2394-400; Aguilar et al., Sci Rep 2018, 8, 9999; Almeida et al., Biochem J 1994, 304 (Pt 3), 793-802). Knowledge of these glycotopes could open doors for biomedical applications such as diagnostics and vaccine development. Unfortunately, the exact structures of these glycotopes are unknown, except for the well-established T. cruzi glycotope Galα1,3Galβ1,4GlcNAcα, which makes up to ˜10% of the parasite's α-Gal-containing mucin O-glycans (Almeida et al., Biochem J 1994, 304 (Pt 3), 793-802). The identification of parasitic α-Gal glycotopes by classic immunoglycomics requires cultivation of large amounts of parasites (sometimes, unfeasible depending upon the parasite life-cycle stage), and isolation and analysis of the cell surface glycans, which require a vast array of analytical methods, such as glycoproteomics, enzymatic and/or chemical release and fluorescent labeling of glycans, glycoprofiling by various high-resolution mass spectrometry (HR-MS)-based approaches, nuclear magnetic resonance (NMR), among others (Chernykh et al., Biochem Soc Trans 2021, 49, 161-86; Wilkinson and Saldova, J Proteome Res 2020, 19, 3890-3905; Borza et al., Curr MolMed 2020, 20, 828-39). This top-down approach is further complicated by the enormous structural diversity of parasite glycocalyces (Rodrigues et al., PLoS Pathog 2015, 11, e1005169). To get around these technical challenges and limitations, the inventors pursued a bottom-up approach, akin to reversing the classical immunoglycomics approach, i.e., reversed immunoglycomics: based on known or potential parasitic α-Gal-glycotopes, glycans of different sizes are synthesized, coupled to a carrier protein, and their antigenicity is interrogated with sera from patients. This reversed immunoglycomics approach can lead to the discovery of novel parasitic α-Gal glycotopes. The inventors selected Old-World cutaneous leishmaniasis (OWCL) as a proof of concept for two reasons: (a) L. major, one of the causative agents of OWCL, abundantly expresses α-Gal-bearing type-II glycoinositolpholipids (GIPLs) whose structures are known (McConville and Ferguson, Biochem J 1993, 294 (Pt 2), 305-24), and are likely to contain immunodominant glycotopes; and (b) the discovery of L. major glycotopes would be impactful because they could serve as biomarkers (BMKs) for OWCL, for which currently no molecular BMKs exist.

Cutaneous leishmaniasis (CL) is an emerging vector-borne infectious disease, caused by different species of the protozoan parasite Leishmania, and is transmitted by infected female sandflies (de Vries et al., Am J Clin Dermatol, 2015, 16, 99-109). It is endemic to the Americas, the Mediterranean basin, the Middle East, and Central Asia, where approximately 700,000 new infections are reported annually and it currently affects approximately 12 million people worldwide (Alvar et al., PLoS One 2012, 7, e35671). CL is usually not life-threatening, but it causes large disfiguring skin ulcers, often associated with secondary infections, that may take months, and in some cases years to heal, and the scarring often leads to social stigma. Furthermore, inadequate treatment of CL may lead to satellite lesions, or nodular lymphangitis (de Vries et al., Am J Clin Dermatol, 2015, 16, 99-109). In the absence of a CL protective vaccine, the only feasible way to control the disease is through chemotherapy, most commonly pentavalent antimonials like sodium stibogluconate (also known as SbV). SbV is one of the most affordable drugs for CL (Vanlerberghe et al., Trop Med Int Health 2007, 12, 274-83), but it is associated with significant side effects, and drug resistance has developed in some regions (Aït-Oudhia et al., Parasitol Res 2011, 109, 1225-32). Alternative drugs include miltefosine, amphotericin B, liposomal amphotericin B, and pentamidine (Santos et al, Parasitol Res 2008, 103, 1-10; Nagle et al., Chem Rev 2014, 114, 11305-34). However, CL treatment responses vary depending on the infecting parasite species (Al-Salem et al., Parasit Vectors 2019, 12, 195). In Northern Africa and the Middle East, the two predominating CL-causing Leishmania species are L. major and L. tropica (Du et al., PLoS Negl Trop Dis 2016, 10, e0004545; Al-Salem et al., Emerg Infect Dis 2016, 22, 931-33; Muhjazi et al., PLoS Negl Trop Dis 2019, 13, e0007827; Youssef et al., Am J Trop Med Hyg 2019, 101, 108-12; Rehman et al., Emerg Infect Dis 2018, 24, 1973-81), each predominating in certain areas. While azole-based drugs (fluconazole, ketoconazole, and itraconazole) (Weina et al., Clin Infect Dis 2004, 39, 1674-80; Goto et. Al., Infect Dis Clin North Am 2012, 26, 293-307), and azithromycin (Goto et al., Expert Rev Anti Infect Ther 2010, 8, 419-33), meglumine antimoniate (Mohebali et al., Acta Trop 2007, 103, 33-40), and miltefosine (Mohebali et al., Acta Trop 2007, 103, 33-40; van Thiel et al., Clin Infect Dis 2010, 50, 80-83) are effective for CL caused by L. major, they are less effective for treating CL caused by L. tropica. On the other hand, the imiquimod analog EAPB0503 (1-(3-methoxyphenyl)-N-methylimidazo[1,2-a]quinoxalin-4-amine) proved to be efficient against L. tropica, but it is inefficient against L. major (El Hajj et al., PLoS Negl Trop Dis 2018, 12, e0006854). This underscores the need for species-specific identification.

The current conflict in the Middle East, particularly in Syria, Afghanistan, and Iraq has resulted in a massive displacement of potentially infected individuals. Many refugees moved into regions where a different Leishmania species may be endemic as compared to the areas of their origin, which complicates the diagnosis by local physicians and can impede treatment. In the last decade, a sharp increase in imported CL cases has been reported in countries of Western Europe due to returning military personnel, labor immigration from endemic zones, and an increased refugee population from the Middle East (Di Muccio et al., PLoS One 2015, 10, e0129418; L. Gradoni, Euro Surveill 2013, 18, 20539; Boecken et al., J Dtsch Dermatol Ges 2011, 9 Suppl 8, 1-51; Herremans et al., Int Health 2010, 2, 42-46; Söbirk et al., Epidemiol Infect 2018, 146, 1267-74; Poeppl et al., Travel Med Infect Dis 2013, 11, 90-94; Pavli et al., Int J Infect Dis 2010, 14, e1032-39). In these countries, many physicians have limited experience with CL, hence belated diagnoses and inappropriate treatment have occurred (Pavli et al., Int J Infect Dis 2010, 14, e1032-39; Antinori et al., Clin Microbiol Infect 2005, 11, 343-46; Manfredi et al., Eur J Epidemiol 2001, 17, 793-95; Kuna et al., Postepy Dermatol Alergol 2019, 36, 104-11).

In the absence of a biomarker (BMK), OWCL is commonly diagnosed by inspection of skin lesions, but the lesions closely resemble other skin conditions, such as atopic dermatitis (eczema), skin cancer, and leprosy (Handler et al., J Am Acad Dermatol 2015, 73, 911-26; Saab et al., J Cutan Pathol 2012, 39, 251-62). Other diagnostic methods include detection of Leishmania amastigotes from skin lesions by microscopy, histopathology, and culture, as well as serology using whole parasite lysate as antigen (de Vries et al., Am J Clin Dermatol 2015, 16, 99-109). However, the limited sensitivity and specificity of these methods do not always accurately diagnose OWCL and cannot differentiate between infecting Leishmania species (de Vries et al., Am J Clin Dermatol 2015, 16, 99-109; de Souza et al., Parasitology 2018, 145, 1938-48; Saab et al., J Cutan Pathol 2012, 39, 251-62). Polymerase chain reaction (PCR) has an extraordinary sensitivity and can differentiate between species, but it is expensive and technologically demanding, and often unavailable in clinical laboratories in developing countries (Handler et al., J Am Acad Dermatol 2015, 73, 911-26; Masmoudi et al., J Dermatol Case Rep 2013, 7, 31-41), where an affordable, species-specific diagnostic test is needed the most.

There is a pressing need for the development of alternative robust and affordable diagnostic tools or biomarkers for the accurate and fast identification of CL suitable for field hospitals and refugee settings. In addition, the differentiation of different Leishmania species is a priority, to inform best drug treatment regimens.

### SUMMARY

Aspects of the present disclosure address needs in the art by providing methods and compositions for identifying and treating Leishmania infection in subjects. Certain aspects are directed to a neoglycoconjugate containing a glycan coupled to a carrier. A neoglycoconjugate can be used to detect antibodies present in a subject infected to exposed to Leishmania.

Certain aspects are directed to a glycoside containing a glycan containing Galpα1,3Galfβ; Galpα1,6Galpα1,3Galβ; and Galpα1,3Galfβ1,3Manpα. In some aspects, the glycoside can have the formula of Galpα1,3Galfβ(CH₂)ₓSH, Galpα1,6Galpα1,3Galfβ(CH₂)ₓSH, or Galpα1,3Galfβ1,3Manpα(CH₂)ₓSH, where x is independently an integer between 1 to 10. In certain aspects, x is 3. In certain aspects, the glycoside can be Galpα1,3Galβ(CH₂)₃SH (FIG. 1A), Galpα1,6Galpα1,3Galβ(CH₂)₃SH (FIG. 1B), or Galpα1,3Galfβ1,3Manpα(CH₂)₃SH (FIG. 1C). In certain aspects, the glycoside can be coupled to a linker, a carrier, or a linker and a carrier.

Certain aspects are directed to a neoglycoconjugate containing a glycan coupled to a carrier, wherein the glycan contains Galpα1,3Galfβ, Galpα1,6Galpα1,3Galfβ, or Galpα1,3Galfβ1,3Manpα. In certain aspects, the carrier is a protein carrier, a peptide carrier, or a nanoparticle carrier. In certain aspects, the protein carrier is an albumin, for example a bovine serum albumin (BSA) or human serum albumin (HSA). The neoglycoconjugate can contain 5 to 50, or at least any one of, equal to any one of, or between any two of 5, 10, 15, 20, 25, 30, 35, 40, 45, and 50 glycans per protein carrier. In certain aspects, the neoglycoconjugate can contain 10 to 30 glycans per carrier or BSA. In certain aspects, the peptide carrier can be a T cell epitope. In certain aspects, the neoglycoconjugate contains a linker connecting the glycoside to the carrier. In some aspects, the linker contains —(CH₂)ₓS—, wherein x is an integer between 1 to 10. In certain aspects, x is 3. In certain aspects, the glycans in the neoglycoconjugate are covalently linked to BSA via the linker, where the linker can be covalently attached to the lysine side chains of BSA. In certain aspects, the linker can have the chemical formula of Formula I:

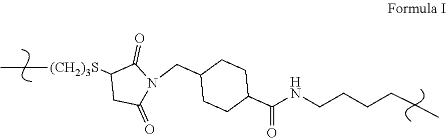

In some respects the neoglycoconjugates can have the chemical formula of the neoglycoconjugates of FIG. 2A (NGP27B or NGP27b), FIG. 2B (NGP28B or NGP28b), or FIG. 2C (NGP30B or NGP30b).

Certain aspects are directed to a method for detecting and/or identifying a parasite. The method can include contacting a blood sample from a subject with a neoglycoconjugate described herein; and detecting binding between the neoglycoconjugate with antibodies in the blood sample that bind a glycan having a terminal, nonreducing α-Gal residue. The subject can be suspected of having or at risk of having (having been recently or is in an endemic area or is suspected of being exposed) cutaneous leishmaniasis (CL). The parasite can be a Leishmania species, Old World or New World. In certain aspects Leishmania species include, but are not limited to L. major, L. tropica, L. aethiopica, L. infantum, L. donovani, L. braziliensis, L. amazonensis, L. panamensis, L. mexicana, L. infantum, or L. guyanensis. In certain aspects a Leishmania species is Leishmania major (L. major) and/or L. tropica. In some respects the parasite can be L. major.

Certain aspects are directed to a method for identifying CL in a subject. The method can include contacting a blood sample from a subject with a neoglycoconjugate described herein, detecting whether the neoglycoconjugate binds with antibodies in the blood sample that bind a glycan having a terminal, nonreducing α-Gal residue; and identifying the subject with CL if binding between the neoglycoconjugate and the antibodies is detected.

Certain aspects are directed to a method for treating CL in a subject. The method can include contacting a blood sample from a subject with a neoglycoconjugate described herein; detecting whether the neoglycoconjugate binds with antibodies in the blood sample that bind a glycan having a terminal αGal; and administering an CL treatment if binding between the neoglycoconjugate and the antibodies is detected. In some aspects an effective amount of a therapeutic agent capable of treating CL can be introduced. In certain aspects, the treatment can be a treatment for a Leishmania species, Old World species and New World species. In certain aspects Leishmania species include but are not limited to L. major, L. tropica, L. aethiopica, L. infantum, L. donovani, L. braziliensis, L. amazonensis, L. panamensis, L. mexicana, L. infantum, or L. guyanensis. In certain aspects a Leishmania species is L. major and/or L. tropica infection. In some respects the treatment can be a treatment for a L. major infection.

In certain aspects, the binding between the neoglycoconjugate and an antibody can be detected using colorimetric, chemiluminescent, or electrochemiluminescent enzyme-linked immunosorbent assay (ELISA). Neoglycoconjugate described herein can specifically bind Leishmania species induced anti-α-Gal Abs and to a less extent to natural anti-α-Gal Abs from healthy individuals. In certain aspects, the neoglycoconjugate can specifically bind L. major, L. tropica, L. aethiopica, L. infantum, L. donovani, L. braziliensis, L. amazonensis, L. panamensis, L. mexicana, L. infantum, or L. guyanensis induced anti-α-Gal Abs. In certain aspects, the neoglycoconjugate can specifically bind L. major induced anti-α-Gal Abs. In certain aspects, the antibodies can be IgG anti-α-Gal antibodies. The antibodies can bind glycan having a terminal, nonreducing α-Gal residue. In certain aspects serum from the blood sample can be contacted with the neoglycoconjugate. In some aspects binding can be determined with respect to a reference level.

The glycan described herein can be a non-natural e.g., synthetic glycan. The subject can be human. A blood sample can include a blood derivative or a blood fraction, e.g., serum or plasma. In some aspects a 1/50 to 1/5000 diluted serum, or at least any one of, equal to any one of, or between any two of 1/50, 1/100, 1/200, 1/300, 1/400, 1/500, 1/600, 1/700, 1/800, 1/900, 1/1000, 1/2000, 1/3000, 1/4000, and 1/5000 diluted serum can be contacted with the neoglycoconjugate. In some aspects, the neoglycoconjugate can be contacted at a concentration of 1 ng/100 μL to 500 ng/100 μL, or at least any one of, equal to any one of, or between any two of 1 ng/100 μL, 3 ng/100 μL, 5 ng/100 μL, 10 ng/100 μL, 15 ng/100 μL, 20 ng/100 μL, 25 ng/100 μL, 30 ng/100 μL, 40 ng/100 μL, 50 ng/100 μL, 100 ng/100 μL, 200 ng/100 μL, 300 ng/100 μL, 400 ng/100 μL, and 500 ng/100 μL.

Certain aspects are directed to a method for inducing an immune response against Leishmania species including but not limited to L. major, L. tropica, L. aethiopica, L. infantum, L. donovani, L. braziliensis, L. amazonensis, L. panamensis, L. mexicana, L. infantum, or L. guyanensis in a human comprising administering a neoglycoconjugate, glycoside and/or glycan described herein, wherein an immune response is generated against the Leishmania species. In some particular aspects the neoglycoconjugate, glycoside and/or glycan are administered to induced immune response against L. major. Certain embodiments are directed to a glycoconjugate-based (e.g., containing neoglycoconjugate, glycoside and/or glycan described herein) a vaccine that induces a long-lasting, full protection against Leishmania (e.g., L. major, L. tropica, L. aethiopica, L. infantum, L. donovani, L. braziliensis, L. amazonensis, L. panamensis, L. mexicana, L. infantum, or L. guyanensis). In certain aspects the glycoconjugate-based vaccine's protection is mediated by B cells. In a further aspect the glycoconjugate-based vaccine's protection is dependent on CD4+ T cells and/or CD8+ T cells. In certain aspects, the glycoconjugate-based vaccine candidates are structurally simple and synthetic. In certain aspects the glycoconjugate-based vaccine candidates can be produced in large scale. In still further aspects, the glycoconjugate-based vaccine candidates are chemically stable.

Other embodiments of the invention are discussed throughout this application. Any embodiment discussed with respect to one aspect of the invention applies to other aspects of the invention as well and vice versa. Each embodiment described herein is understood to be embodiments of the invention that are applicable to all aspects of the invention. It is contemplated that any embodiment discussed herein can be implemented with respect to any method or composition of the invention, and vice versa. Furthermore, compositions and kits of the invention can be used to achieve methods of the invention.

“Effective amount” and “therapeutically effective amount” are used interchangeably herein, and refer to an amount of a therapeutic agent (e.g., small molecule, peptide, antibody etc. drug) effective to achieve a particular biological or therapeutic result such as, but not limited to, the biological or therapeutic results disclosed herein. A therapeutically effective amount may vary according to factors such as the disease state, age, sex, and weight of the individual, and the ability of the therapeutic agent to elicit a desired response in the individual. Such results may include, but are not limited to, the treatment of L. major infection, as determined by any means suitable in the art.

As used herein, the term “subject” refers to any mammal, including both human and other mammals. Preferably, the methods of the present invention are applied to human subjects.

The phrase “specifically binds” or “specifically immunoreactive” to a target refers to a binding reaction that is determinative of the presence of the molecule in the presence of a heterogeneous population of other biologics. Thus, under designated immunoassay conditions, a specified molecule binds preferentially to a particular target and does not bind in a significant amount to other biologics present in the sample. Specific binding of an antibody to a target under such conditions requires the antibody be selected for its specificity to the target. A variety of immunoassay formats may be used to select antibodies specifically immunoreactive with a particular protein. For example, solid-phase ELISA immunoassays are routinely used to select monoclonal antibodies specifically immunoreactive with a protein. See, e.g., Harlow and Lane, Antibodies: A Laboratory Manual, Cold Spring Harbor Press, 1988, for a description of immunoassay formats and conditions that can be used to determine specific immunoreactivity.

As used herein, the term “antigen” is a molecule capable of being bound by an antibody or T-cell receptor. An antigen is additionally capable of inducing a humoral immune response and/or cellular immune response leading to the production of B- and/or T-lymphocytes. The structural aspect of an antigen, e.g., three-dimensional conformation or modification (e.g., phosphorylation), giving rise to a biological response is referred to herein as an “antigenic determinant” or “epitope.” B-lymphocytes respond to foreign antigenic determinants via antibody production, whereas T-lymphocytes are the mediator of cellular immunity. Thus, antigenic determinants or epitopes are those parts of an antigen that are recognized by antibodies, or in the context of an MHC, by T-cell receptors. An antigenic determinant need not be a contiguous sequence or segment of protein and may include various sequences that are not immediately adjacent to one another. In certain embodiments, binding moieties other than antibodies and be engineered to specifically bind to an antigen, e.g., aptamers, avimers, and the like.

The term “antibody” or “immunoglobulin” is used to include intact antibodies and binding fragments/segments thereof. Typically, fragments compete with the intact antibody from which they were derived for specific binding to an antigen. Fragments include separate heavy chains, light chains, Fab, Fab′F(ab′)2, Fabc, and Fv. Fragments/Segments are produced by recombinant DNA techniques, or by enzymatic or chemical separation of intact immunoglobulins. The term “antibody” also includes one or more immunoglobulin chains that are chemically conjugated to, or expressed as, fusion proteins with other proteins. The term “antibody” also includes bispecific antibodies. A bispecific or bifunctional antibody is an artificial hybrid antibody having two different heavy/light chain pairs and two different binding sites. Bispecific antibodies can be produced by a variety of methods including fusion of hybridomas or linking of Fab′ fragments. See, e.g., Songsivilai and Lachmann, Clin Exp Immunol 1990, 79, 315-21; Kostelny et al., J. Immunol 1992, 148, 1547-53. “Antibody” refers to all isotypes of immunoglobulins (IgG, IgA, IgE, IgM, IgD, and IgY) including various monomeric and polymeric forms of each isotype, unless otherwise specified.

The reference level may comprise data obtained at the same time (e.g., in the same hybridization experiment) as the patient's individual data or may be a stored value or set of values e.g., stored on a computer, or on computer-readable media. If the latter is used, new patient data for the selected marker(s), obtained from initial or follow-up samples, can be compared to the stored data for the same marker(s) without the need for additional control experiments.

The terms “treating” or “treatment” refer to any success or indicia of success in the attenuation or amelioration of an injury, pathology or condition, including any objective or subjective parameter such as abatement, remission, diminishing of symptoms or making the injury, pathology, or condition more tolerable to the patient, slowing in the rate of degeneration or decline, making the final point of degeneration less debilitating, improving a subject's physical or mental well-being, or prolonging the length of survival. The treatment or amelioration of symptoms can be based on objective or subjective parameters, including the results of a physical examination, neurological examination, and/or psychiatric evaluations.

The use of the word “a” or “an” when used in conjunction with the term “comprising” in the claims and/or the specification may mean “one,” but it is also consistent with the meaning of “one or more,” “at least one,” and “one or more than one.”

Throughout this application, the term “about” is used to indicate that a value includes the standard deviation of error for the device or method being employed to determine the value.

The use of the term “or” in the claims is used to mean “and/or” unless explicitly indicated to refer to alternatives only or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

As used herein, the terms “comprises,” “comprising,” “includes,” “including,” “has,” “having,” “contains”, “containing,” “characterized by” or any other variation thereof, are intended to encompass a non-exclusive inclusion, subject to any limitation explicitly indicated otherwise, of the recited components. For example, a chemical composition and/or method that “comprises” a list of elements (e.g., components or features or steps) is not necessarily limited to only those elements (or components or features or steps) but may include other elements (or components or features or steps) not expressly listed or inherent to the chemical composition and/or method.

As used herein, the transitional phrases “consists of” and “consisting of” exclude any element, step, or component not specified. For example, “consists of” or “consisting of” used in a claim would limit the claim to the components, materials or steps specifically recited in the claim except for impurities ordinarily associated therewith (i.e., impurities within a given component). When the phrase “consists of” or “consisting of” appears in a clause of the body of a claim, rather than immediately following the preamble, the phrase “consists of” or “consisting of” limits only the elements (or components or steps) set forth in that clause; other elements (or components) are not excluded from the claim as a whole.

As used herein, the transitional phrases “consists essentially of” and “consisting essentially of” are used to define a chemical composition and/or method that includes materials, steps, features, components, or elements, in addition to those literally disclosed, provided that these additional materials, steps, features, components, or elements do not materially affect the basic and novel characteristic(s) of the claimed invention. The term “consisting essentially of” occupies a middle ground between “comprising” and “consisting of”.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### DESCRIPTION OF THE DRAWINGS

The following drawings form part of the present specification and are included to further demonstrate certain aspects of the present invention. The invention may be better understood by reference to one or more of these drawings in combination with the detailed description of the specification embodiments presented herein.

FIG. 1A-1C. Mercaptopropyl glycosides of Galpα1,3Galfβ (G27_{SH}) (A), Galpα1,3Galfβ1,3Manα (G30_{SH}) (B), and Galpα1,6Galpα1,3Galfβ (G28_{SH}) (C).

FIG. 2A-2C. Type-II GIPL-based neoglycoproteins NGP27B (A), NGP28B (B), and NGP30B (C).

FIG. 3: Synthesis of mercaptopropyl glycosides of FIG. 1A to 1C. (a) TMS-OTf, DCM, 0° C. to rt, 1 h, MS 4 Å (50-67%); (b) HF-pyridine, THF, 0° C. then rt, 1 h (65-88%); (c) TFA/H₂O/DCM 1:1:10, rt, 15 min (70-90/o); (d) AcSH, AIBN or DPAP, THF, UV light (350 nm), 6-12 h (85-93%); (e) NaOMe, MeOH, rt, 2 h (quant.); (f) NIS, AgOTf, DCM, 0° C. to rt, 45 min, MS 4 Å (47%); and (g) HF-pyridine (1.2 equiv.), THF, 0° C., 6 h, or HF-pyridine (excess), THF, 0° C., 2 h (60%).

FIG. 4: Conjugation of glycans and maleimide-derivatized BSA 14 to produce NGPs. (a) TCEP.HCl, phosphate buffer pH 7.2, rt, 2 h. The average number of glycans per BSA molecule was determined by matrix-assisted laser desorption/onization time-of-flight mass spectrometry (MALDI-TOF-MS).

FIG. 5: Cross-titrations of NGP27b, NGP28b, and NGP30b, as well as 2-ME-BSA (2-MEb), used as a negative control, with pools of patient sera with active L. major or L. fropica infection, and pooled sera of healthy individuals from Saudi Arabia.

FIG. 6A-6C: (A) Chemiluminescent ELISA reactivity of NGP27b, NGP28b, and NGP30b with sera from individual patients with L. major or L. tropica infection, or with heterologous disease. (B, C) Sera (at 1:800 dilution) from individual patients with active L. major or L. tropica infection, or with heterologous disease (with other skin conditions) were evaluated against NGP27b, NGP28b, and NGP30b, each at 25 ng/well. Chemiluminescent ELISA was performed as follows. Levels of Leishmania-specific anti-α-Gal IgG antibodies in human sera was determined by chemiluminescent ELISA essentially as previously described (Al-Salem et al., Parasilology 2014, 141, 1898-903; Subramaniam et al., Parasitology 2018, 145, 1758-64) with modifications. The serum dilutions and concentrations of NGPs (NGP27b, NGP28b, and NGP30b) and control antigen (2-MEb) varied between assays, but the overall immunoassay steps remained the same. Briefly, MaxiSorp white 96-well microplates (Nunc, Thermo Fisher Scientific) were coated with the appropriate NGP in 0.2 M carbonate-bicarbonate buffer, pH 9.6 (CBB), at 4° C., 16 h, at concentrations determined through antigen and serum cross-titration experiments. The free sites on the microplate wells were blocked with 200 μL/well PBS-1% BSA (PBS-B), and incubated with sera from patients with L. major or L. tropica CL infection, heterologous diseases, or from healthy individuals, diluted in PBS-B as indicated in each experiment. Anti-human IgG (H+L) biotin conjugate (1:10,000 dilution, Cat #31770, Thermo Fisher Scientific) was added (50 μL/well). The biotin complex was recognized by adding 50 μL/well of Pierce High Sensitivity NeutrAvidin-HRP (1:5,000 dilution, Cat #31030, Thermo Fisher Scientific). In all incubation steps, plates were incubated for 1 h at 37° C. Between incubation steps, plates were washed three times with 200 μL/well PBS-0.05% Tween 20 (PBS-T). Following addition of SuperSignal™ ELISA Pico Chemiluminescent Substrate (Thermo Fisher Scientific, 37070), diluted 1:8 (v/v) in CBB, the luminescence was immediately measured, as relative luminescence units (RLUs), using a FLUOstar™ Omega multi-mode microplate reader (BMG LabTech, Ortenberg, Germany). Positive and negative controls for each microplate were included in triplicate or duplicate. The negative control consisted of a serum pool of ten randomly selected healthy individuals from Saudi Arabia. The positive control consisted of a serum pool of ten randomly selected L. major CL patients. In preliminary immunoassays, to determine the nonspecific background reactivities, we also included negative control wells lacking the antigen, primary antibody (serum), secondary conjugate (biotinylated anti-human IgG antibody), or NeutrAvidin-HRP. The average RLU was taken for the negative and positive controls and subtracted from the average of the experimental sample, tested in duplicate or triplicate, to control for nonspecific/background signal from the reagents. The cutoff of each immunoassay microplate was determined using the method described by Frey et al. (J Immunol Methods 1998, 221, 35-41) Briefly, the upper prediction limit was defined, expressed as the standard deviation (SD) multiplied by a factor (1) (SDf), calculated based on the Student t-distribution, according to the number of negative control (NC) replicates in each plate and a confidence level (1−α) of 95%. Therefore, the cutoff value in each was calculated as the NC RLU mean+SDf Since the titer of each ELISA was defined as the ratio of the experimental sample's average RLU value to the cutoff value. A serum sample was considered positive when its titer was equal to or higher than 1.000 and negative when the titer was lower than 1.000. (a) Grouped scatter plot analysis of sera from L. major or L. tropica infections, or heterologous diseases with NGP27b, NGP28b, or NGP30b. Cᵢ, initial cutoff value (titer=1.000). C_{L.t.}, adjusted cutoff value, calculated based on the receiver operating characteristic (ROC) and two-graph (TG)-ROC curve analysis data (b and c), for the comparison between sera from L. major vs. L. tropica infections. C_{Het.}, adjusted cutoff value for the comparison between sera from L. major infection vs. heterologous diseases. Statistical analysis: nonparametric Mann-Whitney test; significance level: p<0.05. ****, p<0.0001; ns, non-significant. (b) Receiver operating characteristic (ROC) curves for NGP27b, NGP28b, and NGP30b, comparing the reactivity of sera from L. major vs. L. tropica infections (top row) or sera from L. major infections vs. heterologous diseases (bottom row), using the data depicted in the scatter plots (a). AUC, area under the curve is indicated (grey area). In parenthesis, 95% confidence interval values are indicated. (c) TG-ROC curve analysis was performed by plotting the ROC data (b) for sensitivity (Se) and specificity (Sp), as described by Greiner et al. (Prev Vet Med 2000, 45, 23-41). Shaded area indicates the cutoff value interval where Se or Sp could reach 100%. The Se (black) and Sp (grey) raw data points are represented as fine lines, whereas the fitted data are indicated as thick lines. Vertical continuous black line, original titer cutoff value (C, =1.000); vertical dotted grey line, adjusted cutoff value for the comparison L. major infections vs. heterologous diseases; vertical dashed gray line, adjusted cutoff value for the comparison L. major vs. L. tropica infections. The adjusted cutoff values are indicated in grey on top of each graph.

FIG. 7. Algorithm for discriminating L. major infection from L. tropica infection, and heterologous disease, using NGP27b and NGP30b, sequentially, in chemiluminescent ELISA.

FIG. 8. Synthesis of the mercaptopropyl disaccharide G27_{SH}.

FIG. 9. Synthesis of the mercaptopropyl trisaccharide G28_{SH}.

FIG. 10. Chemiluminescent ELISA reactivity of 2-Meb with sera from patients with L. major or L. tropica infection, heterologous disease, or from healthy individuals. Grouped scatter plot analysis of individual (L. major CL, n=81; heterologous diseases, n=24; and L. tropica, n=15) or pooled sera (at 1:800 dilution) from patients with active L. major (n=8) or L. tropica (n=8) infection, or from healthy individuals (n=8) from England, UK, were evaluated by chemiluminescent ELISA against 2-Meb (negative control antigen) at 25 ng/well. Each point represents the mean of triplicate or duplicate of each pool, tested in separate microplate reactions (total=8), performed at the same or different days. The immunoassay was performed as described in FIG. 6. The horizontal line indicates the initial cutoff value (Cᵢ, titer=1.000). Statistical analysis: nonparametric Mann-Whitney test; significance level: p<0.05. *, p<0.05; **, p<0.001; ns, non-significant.

FIG. 11A-11D. Neoglycoproteins synthesized and used in this study. (A) Schematic representation of type-2 GIPLs 1-3 of L. major. The terminal glycan moiety (G29, G30, or G28) targeted for chemical synthesis in each GIPL is indicated. Galp, galactopyranose; Galf, galactofuranose; Man, mannopyranose; GlcN, glucosamine; myo-Ins, myo-inositol; P, phosphate; PI, phosphatidylinositol. (B) Schematic representation of the synthesis of NGP29b containing the type-2 GIPL-1 terminal, nonreducing glycotope Galfβ1,3Manα. TCEP-HCl, Tris (2-carboxyethyl) phosphine hydrochloride; linker, 4-(succinimidomethyl)cyclohexane-1-carboxy group. The same conjugation was used for the synthesis of NGP30b and NGP28b (Montoya et al., JACS Au. 2021, 1(8):1275-1287). (C) Representative MALDI-TOF-MS spectrum of NGP29b to confirm the covalent conjugation of the glycan units to the carrier protein, as recently described (Montoya et al., Molecules. 2022, 27(2):411). The same quality-control procedure was used for NGP30b and NGP28b, as previously described (Montoya et al., JACS Au. 2021, 1(8):1275-1287). Singly-([BSA]⁺ and [NGP29b]⁺) and doubly-charged ([BSA]⁺² and [NGP29b]⁺²) ions of BSA and NGP29b are indicated. The number of glycan units (=30) covalently attached to the BSA moiety is indicated. m/z, mass to charge ratio. (D) Composition of the synthetic NGP29b, NGP30b, and NGP28b. For simplicity, the linker group between the thiopropyl glycan derivative and the lysine residue, shown in B, is not indicated.

FIG. 12. Cross-titration of NGPs with serum pools from CL or ML clinical forms of tegumentary leishmaniasis, Chagas disease, and nonendemic controls. cELISA tests were performed with NGP29b, NGP28b, or NGP30b at concentrations ranging from 50 to 3.13 ng/well, using pool of sera (n=15, each) from patients with active CL or ML, caused by L. braziliensis. Pools of sera obtained from patients with chronic Chagas disease (CD) (n=15), and from nonendemic controls (NEC) (n=15) were also evaluated. Each point represents the mean of duplicate values of the relative luminescence units (RLU) obtained for each sample and bars indicate SD. Statistical analysis: two-way Anova with main effects only and Dunnett's multiple comparison test (with individual variances computed for each comparison). The CL, ML, or CD curve was compared with the NEC curve, at 1:400 and 1:800 serum pool dilution. *p<0.05, **p<0.01,****p<0.0001; statistically non-significant differences are not shown.

FIG. 13. Normalized IgG response of sera from patients with tegumentary leishmaniasis (TL) to GIPL-1-based NGP29b (A) and GIPL-3-based NGP28b (B). cELISA immunoassays were performed using NGPs at 5 ng/well and serum samples (1:800 dilution) from all TL samples (n=80), with different clinical forms (CL; n=17; ML n=16; DL, n=16; and SC, n=31) plotted separately; Chagas disease (CD, n=16); and all non-TL, seemingly healthy controls (NEC+EC; n=33), also plotted separately (EC, n=15; and NEC, n=18). Each point represents the mean of triplicate relative luminescence units (RLU) values normalized to NEC serum pools. The cutoff value (cELISA titer=1.000), calculated as described in Materials and Methods, is indicated by the continuous green line. Data are represented as violin plots (truncated) of individual points, with median (thick black line) and interquartile range (dotted black lines) values indicated. *p<0.05, **p<0.01, ****p<0.0001, Kruskal Wallis followed by Dunn's multiple comparison tests. Statistically non-significant differences between serum groups are not shown.

FIG. 14. Receiver-operating characteristic (ROC) curves for NGP29b and NGP28b comparing the reactivity of sera from total TL patients or CL, ML, DL, SC, or CD patients versus control sera from endemic (EC) and nonendemic (NEC) individuals, using cELISA titers normalized to NECs. Area under the curve (AUC) is indicated in the gray area, and 95% confidence interval (CI) values are indicated in parentheses.

FIG. 15. Seroreactivity to NGP28b of TL patients, before and after treatment. Sera from CL (n=17) or ML (n=16) patients, obtained before and 90 days after standard Sb^{V} treatment, were probed by cELISA with NGP28b (5 ng/well). Each point represents the mean of triplicate RLU values normalized to a pool of sera from seemingly healthy NEC individuals. p Values were calculated using Wilcoxon matched-pairs test. Significance level: p<0.05.

FIG. 16. Receiver-operating characteristic (ROC) curves for NGP28b comparing the reactivity of sera from total TL patients, or CL, ML, DL, or SC patients versus CD patients, using cELISA titers normalized to NECs. The AUC is indicated in the gray area, and 95% CI values are indicated in parentheses.

FIG. 17. Two-graph (TG)-ROC curve analysis was performed for by plotting the ROC data for sensitivity (Se, black lines) and specificity (Sp, purple lines) against NGP28b for all TL patients or CL, ML, DL, or SC patients versus EC+NEC individuals. The Se and Sp raw data points are represented as thick lines, whereas the best-fitted data are indicated as smooth fine lines. Shaded area indicates the cELISA titer interval where Se or Sp could reach 100%. Vertical black line, initial cELISA titer cutoff value (Cᵢ=1.000); vertical dotted gray line, adjusted cELISA titer value (Ca, gray number on top) for the comparison of TL, CL, ML, DL, or SC patients versus controls (EC+NEC). Note that, for TL, CL, and ML patients, no Cavalue was generated and the Cᵢ=1.000 remained as the cutoff.

### DESCRIPTION

The glycocalyx of Leishmania parasites is predominantly composed of glycoinositolphospholipids (GIPLs), as well as lipophosphoglycans (LPGs) and glycosylphosphatidylinositol (GPI)-anchored proteins, such as GP63 and proteophosphoglycans (PPGs) (McConville and Ferguson, Biochem J 1993, 294 (Pt 2), 305-24; Cabezas et al., Org Biomol Chem 2015, 13, 8393-404; Schneider et al., Biochem J 1994, 304 (Pt 2), 603-9; Forestier et al., Front Cell Infect Microbiol 2014, 4, 193; Guha-Niyogi et al., Glycobiology 2001, 11, 45R-59R). L. major synthesizes highly abundant type-II GIPLs (GIPL-1, GIPL-2, and GIPL-3), which contain 0-galactofuranosyl (0-Galf) (GIPL-1) or α-galactopyranosyl (α-Galp) (GIPL-2 and -3) residue at their terminal, nonreducing ends (McConville and Ferguson, Biochem J 1993, 294 (Pt 2), 305-24). There is a considerable body of evidence that these unusual sugars expressed in L. major and in other pathogens, such as Trypanosoma cruzi, fungi, and enterobacteria, are highly immunogenic to human hosts because they are either cryptic or not normally expressed (α-Galp), or entirely absent (0-Galf) on human cells (McConville and Ferguson, Biochem J 1993, 294 (Pt 2), 305-24; McConville and Bacic, J Biol Chem 1989, 264, 757-66; McConville et al., J Biol Chem 1990, 265, 7385-94; Avila et al., J Clin Microbiol 1991, 29, 2305-12; Rosen et al., Mol Biochem Parasitol 1988, 27, 93-9; Schnaidman et al., J Protozool 1986, 33, 186-91; Travassos and Almeida, Springer Semin Immunopathol 1993, 15, 183-204; Galili, The Natural Anti-Gal Antibody As Foe Turned Friend In Medicine 2018, 1-18; Wilkinson, Prog. Lipid Res. 1996, 35, 283-343). On the other hand, the main GIPLs of L. tropica belong to the α-mannose-terminating GIPLs iM2, iM3, and iM4 (Schneider et al., Biochem J 1994, 304 (Pt 2), 603-9). The inventors contemplate that terminal oligosaccharide partial structures of type-II GIPLs of L. major may be immunodominant glycotopes that are serologically exploitable as diagnostic BMKs specifically for OWCL caused by L. major, and distinguishable from OWCL caused by L. tropica, as well as from non-OWCL dermatological (heterologous) diseases such as atopic dermatitis (eczema), and bacterial and fungal infections. To address this, specific L. major-derived glycan partial structures needed to be synthesized and immunologically evaluated.

The inventors have contemplated that terminal oligosaccharide partial structures of the GIPLs of L. major may be immunodominant glycotopes that are serologically exploitable as diagnostic BMKs specifically for CL caused by L. major, and distinguishable from CL caused by L. tropica, or from heterologous skin diseases (e.g., atopic dermatitis, skin cancer, leprosy, and bacterial and fungal infections) that can be misdiagnosed as CL. Specific L. major-derived glycan partial structures were synthesized and immunologically evaluated.

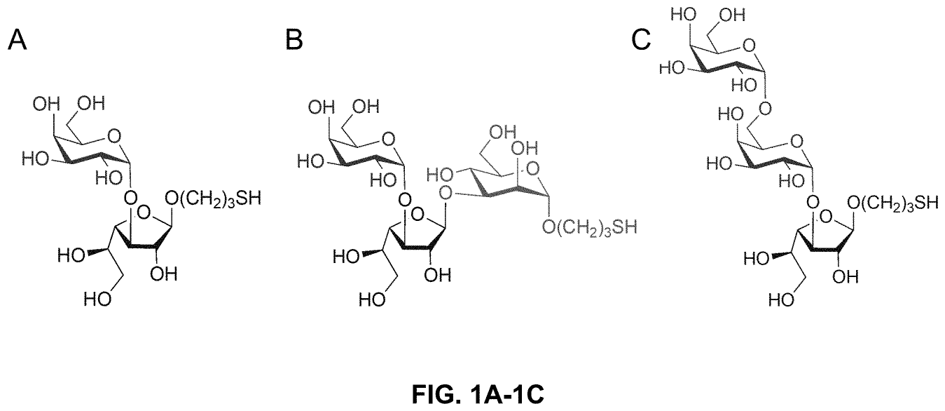

Reversed immunoglycomics was used as a combined chemical and serological approach to discover specific glycan BMKs suitable for the diagnosis of L. major infection and for the distinction from L. tropica infection using synthetic cell surface glycans of the parasite. Instead of isolating glycoconjugates of cultivated parasites and studying their glycoimmunology, this classical immunoglycomics approach was reversed by probing synthetic partial structures of known cell surface glycans for antibody responses by chemiluminescent ELISA. Sera of CL patients from Saudi Arabia contain elevated levels of IgG anti-α-Gal antibodies, that partially recognize simple α-Gal-containing saccharides, but a specific α-Gal biomarker remained elusive. Therefore, focus was shifted to α-galactopyranose (α-Galp or α-Gal)- and β-galactofuranose (β-Galf)-containing saccharides of the terminal glycan portions of type-II GIPLs of L. major. Syntheses for three type-II GIPL partial structures with a chemical handle at the reducing end allowing for conjugation was performed. Specifically, mercaptopropyl glycosides of the GIPL-2 derived glycans Galpα1,3Galfβ (FIG. 1A); Galpα1,3Galfβ1,3Manpα (FIG. 1B); and the GIPL-3 derived glycan Galpα1,6Galpα1,3Galfβ (FIG. 1C) were synthesized. Conjugation of these oligosaccharides to bovine serum albumin (BSA) produced neoglycoproteins (NGPs), which, unlike the oligosaccharides by themselves, adhere effectively to microtiter plates. These NGPs served as antigens in chemiluminescent ELISA, using the sera of CL patients from Saudi Arabia with confirmed L. major or L. tropica infections.

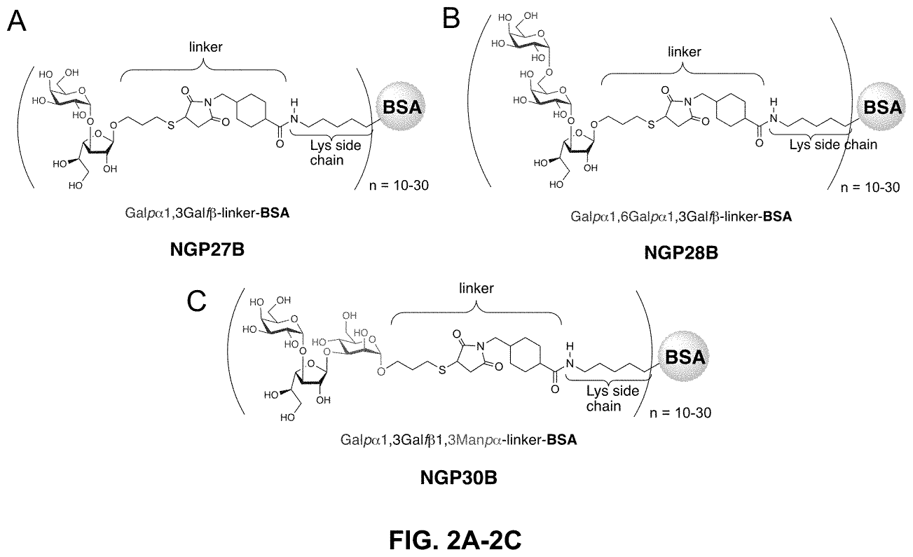

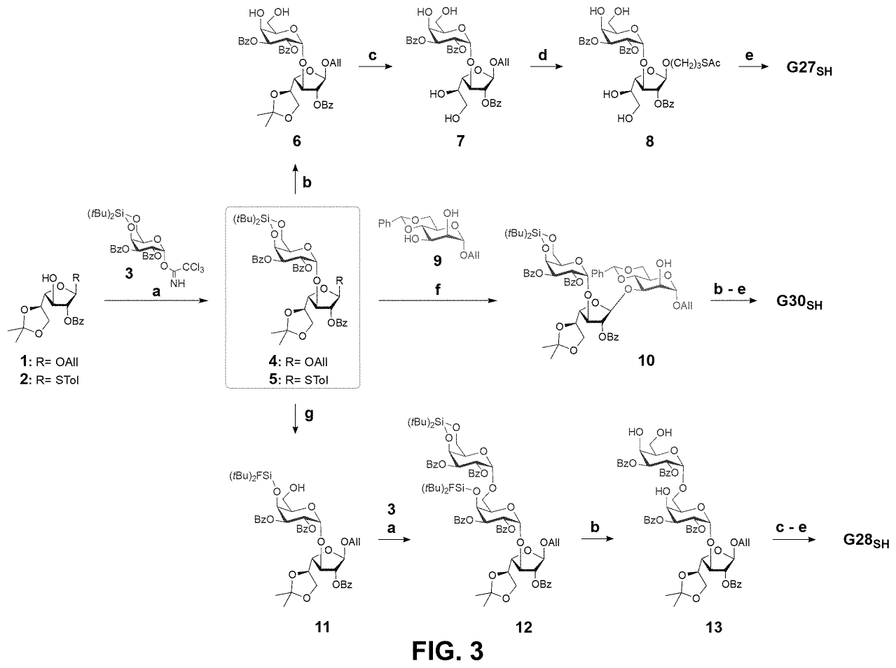

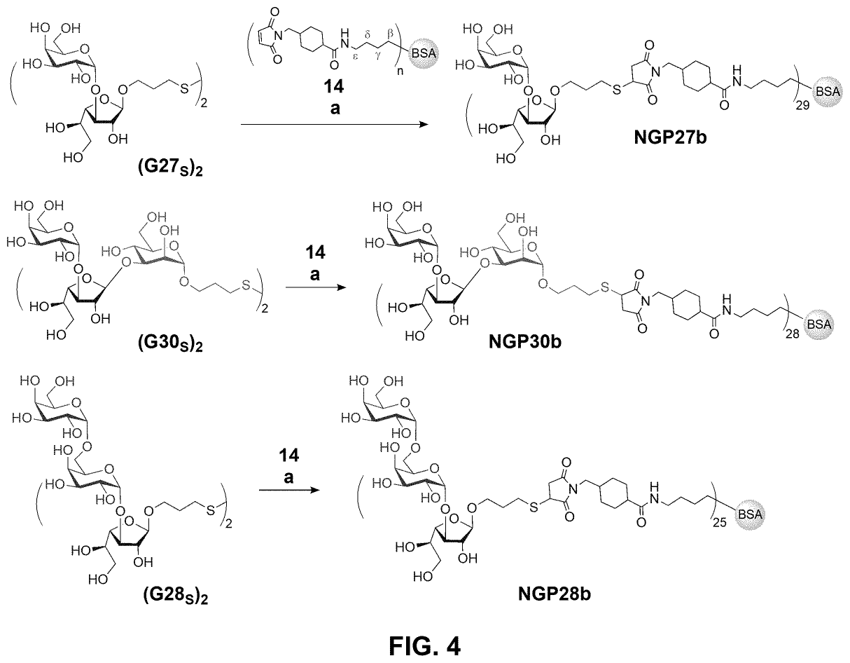

Referring to FIGS. 3 and 4, in certain aspects, the neoglycoconjugates (FIG. 2) containing the glycans of FIG. 1 can be synthesized by (a) the stereoselective α-galactosylation using Kiso's 4:6-di-tert-butylsilylidene-galactosyl trichloroacetimidate donor; (b) an orthogonal protecting group strategy primarily based on acyl, acetal, and silyl groups, and (c) the use of mercaptopropyl glycosides for the conjugation to maleimide-derivatized bovine serum albumin (BSA). The synthesis of GIPL-3 derived trisaccharide 3 also included an unprecedented regioselective ring opening of the 4,6-O-di-tert-butylsilylidene moiety to gain access to a disaccharide acceptor in a single step.

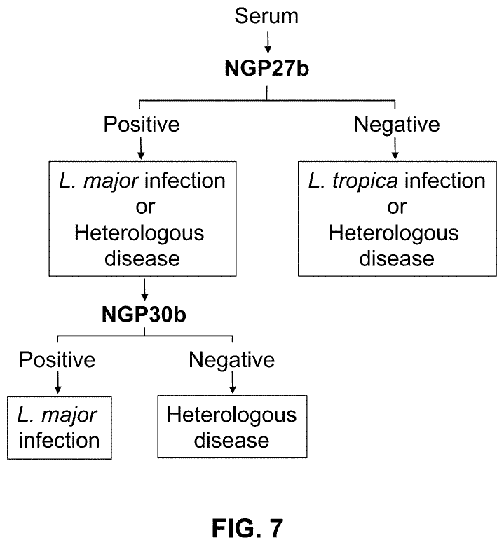

Reversed immunoglycomics and ROC/TG-ROC analysis identified NGP27B (Galpα1,3Gaflβ-BSA) and NGP30B (Galpα1,3Galfβ1,3Manpα-BSA) as the first useful and accurate diagnostic BMKs for CL caused by L. major. NGP27B can distinguish with 95% sensitivity and 100% specificity CL caused by L. major from that caused by L. fropica; whereas NGP30B can distinguish with 88% sensitivity and 100% specificity CL caused by L. major from heterologous diseases. These BMKs can be used in tandem (NGP27B followed by NGP30B), or in parallel (e.g., multiantigen array), and with the assistance of an algorithm (FIG. 7), they can discriminate with 100% specificity CL caused by L. major from CL caused by L. tropica and heterologous diseases. These BMKs can therefore inform the best treatment option for CL.

This BMK discovery approach combined the synthesis of terminal saccharides of cell surface glycoconjugates of L. major that contain immunogenic motifs, and their immunological interrogation by chemiluminescent ELISA using sera of CL patients with L. major or L. tropica infection, as well as NHS of healthy individuals. In some aspects the mercaptopropyl glycosides of Galpα1,3Galfβ (G27_{SH}), and Galpal,6Galpα1,3Galfβ (G28_{SH}), and Galpα1,3Galfβ1,3Manα (G30_{SH}), corresponding to the terminal di- and trisaccharide moieties of type-II GIPLs present in L. major were synthesized using a protecting group strategy. In some aspects acyl, acetal, and/or silyl protecting groups were used. The 4,6-O-DTBS group of Galp can play two roles: (a) It allowed for stereoselective α-galactosylation; and (b) it undergoes a regioselective ring opening reaction producing a new Gal acceptor in a single step suitable for glycosylation at position 6. The mercapto (SH) groups of the glycosides of FIG. 1 allowed for the convenient conjugation to maleimide-derivatized BSA to produce neoglycoconjugates (FIG. 2) which are suitable antigens for chemiluminescent ELISA, or other type of ELISA (e.g., colorimetric or electrochemiluminescent).

GIPL-2 and GIPL-3 are both strongly expressed by L. major. The superiority of NGP30b as a BMK over NGP27b and NGP28b, in the accurate (100% specificity) differential diagnosis of CL caused by L. major from heterologous skin diseases, may be attributed to the presence of an α-mannose moiety at the reducing end. One possible explanation is that the mannose moiety is part of an immunodominant glycotope, another is that the mannose unit affects the conformation of the terminal disaccharide Galpα1,3Galfβ necessary for antibody recognition. The motif α-Gal-1,6-α-Gal present in GIPL-3 may have a greater cross reactivity with NHS due to similar glycan structures in enterobacteria.

### I. Glycoside and Neoglycoconjugates

The neoglycoconjugates comprise a glycan attached to a carrier. The glycan can be attached via linker. In certain aspects the carrier can be a protein, peptide, or nanoparticle. The glycan can be Galpα1,3Galfβ, and Galpα1,6Galpα1,3Galfβ, or Galpα1,3Galfβ1,3Manα.

In one example, BSA was chosen for the generation of neoglycoconjugates because of its large number of conjugation sites per BSA molecule, its solubility properties, and its suitability as a carrier protein (Mäkelä and Seppällä, Handbook of Experimental Immunology. vol. 1. Immunochemistry 1986, 3.1-3.31) and provider of T cell epitopes for the immunization of mice (Atassi et al., Mol Immunol 1982, 19, 313-21), as well as its capability to attach non-covalently to wells of microtiter plates.

Other suitable carrier proteins include human serum albumin (HSA), keyhole limpet hemocyanin (KLH), ovalbumin (OVA), tetanus toxoid (TT), recombinant proteins from L. major containing CD4 and/or CD8 T cell epitopes, Neisseria meningitidis outer membrane protein complex, synthetic peptides, heat shock proteins, pertussis proteins, cytokines, lymphokines, hormones, growth factors, artificial proteins comprising multiple human CD4+ T cell epitopes from various pathogen-derived antigens, protein D from Haemophilus influenzae, pneumolysin or its non-toxic derivatives, pneumococcal surface protein PspA, iron-uptake proteins, toxin A or B from Clostridium difficile, recombinant Pseudomonas aeruginosa exoprotein A (rEPA) and the like.

In certain aspects the carrier can include one or more T-cell epitope. T cell epitopes, e.g., CD4+ T helper cell epitopes (Etlinger et al., Science 1990, 249, 423-5), are peptides that can induce T cell help and are known in the art. Epitopes that are useful in the present methods and compositions include those from diphtheria toxoid (DT), tetanus toxin (TI), Plasmodium falciparum circumsporozite surface protein, hepatitis B surface antigen, hepatitis B nuclear core protein, H. influenzae matrix protein, H. influenzae haemagglutinin, group B N. meningitidis outer membrane protein complex (OMPC), the pneumococcal toxin pneumolysin, and heat shock proteins, including those recombinantly produced and detoxified variants thereof.

In certain aspects the T cell epitope may not include any lysine residues internally, but will be modified to include at least one lysine residue at an end, e.g., at the C terminus. In some embodiments, there is only one lysine residue at the C terminus or at the N terminus. In some embodiments, there will also be one or more amino acids between the carrier peptide sequence and the glycan component of the neoglycoconjugate, i.e., an amino acid spacer sequence. Such spacer sequences can be any amino acid and will generally be flexible and have small R groups, to avoid steric hindrance and allow for optimal positioning of the linked carbohydrate for presentation to T cells and access of the peptide epitope to bind to an effector cell. Exemplary amino acids suitable for inclusion in the linker include glycine, alanine, and serine. In certain aspects the spacer does not contain lysine residues. In certain embodiments two or more carrier peptides are linked or cross-linked with two or more other carrier peptides.

In other embodiments the carrier may be a nanoparticle carrier. The glycan or glycoside can be linked to biocompatible nanoparticles, with or without a linker or spacer between the glycan and the nanoparticle. The nanoparticles useful in the methods and compositions described herein are made of materials that are (i) biocompatible, i.e., do not cause a significant adverse reaction in a living animal when used in pharmaceutically relevant amounts; (ii) feature functional groups to which the binding moiety can be covalently attached, (iii) exhibit low non-specific binding of interactive moieties to the nanoparticle, and (iv) are stable in solution, i.e., the nanoparticles do not precipitate. The nanoparticles can be monodisperse (a single crystal of a material, e.g., a metal, per nanoparticle) or polydisperse (a plurality of crystals, e.g., 2, 3, or 4, per nanoparticle).

A number of biocompatible nanoparticles are known in the art, e.g., organic or inorganic nanoparticles. Liposomes, dendrimers, carbon nanomaterials and polymeric micelles are examples of organic nanoparticles. Quantum dots can also be used. Inorganic nanoparticles include metallic nanoparticle, e.g., Au, Ni, Pt and TiO₂ nanoparticles. Magnetic nanoparticles can also be used, e.g., spherical nanocrystals of 10-20 nm with a Fe²⁺ and/or Fe³⁺ core surrounded by dextran or PEG molecules. In some embodiments, colloidal gold nanoparticles are used, e.g., as described in U.S. Pat. Nos. 7,060,121 and 7,232,474.

The linkers or spacers can be polymer or amino acid linkers. The linker or spacer will comprise a functional group that provide for attachment to the glycan and another functional group that provides for attachment to the carrier. A variety of linker molecules may be used, using conventional procedures. The linker can be any of a wide array of linking groups. Alternatively, the linker may be a single bond or a “zero order linker.”

Said linker molecule is advantageously a homobifunctional or heterobifunctional molecule, for example adipic dihydrazide, ethylenediamine, cystamine, N-succinimidyl-3-(2-pyridyldithio) propionate (SPDP), N-succinimidyl-[N-(2-iodoacetyl)]-s-alanyl propionate-propionate (SIAP), succinimidyl-4-(N-maleimido-methyl)cyclohexane-1 carboxylate (SMCC), 3,3′-dithiodipropionic acid. In certain aspects the linker or spacer is a water-soluble polymer, and in one embodiment, the water-soluble polymer comprises poly(ethylene glycol).

### II. Immunogenic Compositions and Uses Thereof

The glycosides (FIG. 1A-C) and/or neoglycoconjugate (FIG. 2A-C) can be used as diagnostic or prognostic biomarkers, vaccines, treating or detecting for parasitic diseases, such as cutaneous leishmaniasis (CL). CL can be caused by Leishmania, species such as L. major, L. tropica, L. aethiopica, L. infantum, L. donovani, L. braziliensis, L. amazonensis, L. panamensis, L. mexicana, L. infantum, or L. guyanensis. In certain aspects Leishmania species are Leishmania major (L. major) and/or Leishmania tropica (L. tropica). In some particular aspects, the parasite can be L. major. In certain aspects, the glycosides and/or the neoglycoconjugate can be used diagnostic or prognostic biomarkers, vaccines, treating or detecting for CL caused from L. major infection.

An “antigenic determinant” is, unless otherwise indicated, a molecule that is able to elicit an immune response in a particular animal or species. Antigenic determinants include, for example, carbohydrate moieties, such as glycans. In certain aspects an antigenic determinant that is a carbohydrate can be referred to as a “glycotope”.

Certain embodiments are directed to immunogenic compositions and methods comprising a conjugate containing a glycan such as Galpα1,3Galfβ, Galpα1,6Galpα1,3Galfβ, or Galpα1,3Galfβ1,3Manpα. The conjugate can contain a peptide or protein that has one or more glycan moieties covalently attached, either directly or by a linker.

As used herein, “prophylactic” and “preventive” vaccines are vaccines that are designed and administered to prevent or reduce the probability of infection, disease, and/or any related sequela(e) caused by or associated with a pathogenic organism, such as a Leishmania, e.g., L. major.

As used herein, “therapeutic” vaccines are vaccines that are designed and administered to patients already infected with a pathogenic organism. Therapeutic vaccines are used to prevent and/or treat the development of disease in these infected individuals.

As used herein the phrase “immune response” or its equivalent “immunological response” refers to a humoral (antibody-mediated), cellular (mediated by antigen-specific T cells or their secretion products) or both humoral and cellular response directed against an epitope of the invention in a subject or a donor subject. A donor subject is one in which an antibody is generated and isolated, the isolated antibody is then administered to a second subject. Treatment or therapy can be an active immune response induced by administration of immunogen or a passive therapy affected by administration of antibody, antibody-containing material, or vaccine-primed B and/or T cells.

For purposes of this specification and the accompanying claims the terms “epitope” and “antigenic determinant” are used interchangeably to refer to a site on an antigen to which B and/or T cells respond or recognize.

Embodiments described herein include methods for preventing or ameliorating parasite infections. As such, the invention contemplates vaccines for use in both active and passive immunization embodiments. Immunogenic compositions, proposed to be suitable for use as a vaccine, may be prepared from immunogenic glycans and glycan conjugates.

Typically, such vaccines are prepared as injectables, either as liquid solutions or suspensions: solid forms suitable for solution in or suspension in liquid prior to injection may also be prepared. The preparation may also be emulsified. The active immunogenic ingredient is often mixed with excipients that are pharmaceutically acceptable and compatible with the active ingredient. Suitable excipients are, for example, water, saline, dextrose, glycerol, ethanol, or the like and combinations thereof. In addition, if desired, the vaccine may contain amounts of auxiliary substances such as wetting or emulsifying agents, pH buffering agents, or adjuvants that enhance the effectiveness of the vaccines.

Vaccines may be conventionally administered parenterally, by injection, for example, either subcutaneously or intramuscularly. Formulations can include such normally employed excipients and contain about 10% to about 95% of active ingredient, preferably about 25% to about 70%.

The glycans and glycan-conjugates may be formulated into a vaccine as neutral or salt forms. Pharmaceutically-acceptable salts include the acid addition salts (formed with the free amino groups of the peptide) and those that are formed with inorganic acids such as, for example, hydrochloric or phosphoric acids, or such organic acids as acetic, oxalic, tartaric, mandelic, and the like.

Typically, vaccines are administered in a manner compatible with the dosage formulation, and in such amount as will be therapeutically effective and immunogenic. The quantity to be administered depends on the subject to be treated, including the capacity of the individual's immune system to synthesize antibodies and the degree of protection desired. Precise amounts of active ingredient required to be administered depend on the judgment of the practitioner. However, suitable dosage ranges are of the order of several hundred micrograms of active ingredient per vaccination. Suitable regimes for initial administration and booster shots are also variable but are typified by an initial administration followed by subsequent inoculations or other administrations.

In certain instances, it will be desirable to have multiple administrations of the vaccine, e.g., 2, 3, 4, 5, 6 or more administrations. The vaccinations can be at 1-, 2-, 3-, 4-, 5-, 6-, 7-, 8-, to 5-, 6-, 7-, 8-, 9-, 10-, 11-, and 12-week intervals, including all ranges there between. Periodic boosters at intervals of 1-5 years will be desirable to maintain protective levels of the antibodies. The course of the immunization may be followed by assays for antibodies against the antigens.

Carriers. A given composition may vary in its immunogenicity. It is often necessary therefore to boost the host immune system, as may be achieved by coupling an antigen to a carrier. Carriers include, but are not limited to keyhole limpet hemocyanin (KLH) and bovine serum albumin (BSA). Other albumins such as ovalbumin, mouse serum albumin, human serum albumin, or rabbit serum albumin can also be used as carriers. Means for conjugating an antigen to a carrier protein are well known in the art and include glutaraldehyde, m-maleimido benzoyl-N-hydroxysuccinimide ester (MBS), carbodiimide, and bis-diazotized benzidine (BDB).

Adjuvants. The immunogenicity of a composition can be enhanced by the use of non-specific stimulators of the immune response, known as adjuvants. Suitable adjuvants include all acceptable immunostimulatory compounds, such as cytokines, toxins, or synthetic compositions. A number of adjuvants can be used to enhance an antibody response against an antigen. Adjuvants can (1) trap the antigen in the body to cause a slow release; (2) attract cells involved in the immune response to the site of administration; (3) induce proliferation or activation of immune system cells; or (4) improve the spread of the antigen throughout the subject's body.

Adjuvants include, but are not limited to, oil-in-water emulsions, water-in-oil emulsions, mineral salts, polynucleotides, and natural substances. Specific adjuvants that may be used include IL-1, IL-2, IL-4, IL-7, IL-12, 7-interferon, GMCSP, BCG, aluminum salts, such as aluminum hydroxide or other aluminum compound, muramyl dipeptide (MDP) compounds, such as thur-MDP and nor-MDP, CGP (MTP-PE), lipid A, and monophosphoryl lipid A (MPLA). RIBI, which contains three components extracted from bacteria, MPL, trehalose dimycolate (TDM), and cell wall skeleton (CWS) in a 2% squalene/Tween 80 emulsion.

Various methods of achieving adjuvant affect for the vaccine includes use of agents such as aluminum hydroxide or phosphate (alum), commonly used as about 0.05 to about 0.1% solution in phosphate buffered saline, admixture with synthetic polymers of sugars (Carbopol®) used as an about 0.25% solution, aggregation of the protein in the vaccine by heat treatment with temperatures ranging between about 70° to about 101° C. for a 30-second to 2-minute period, respectively. Aggregation by reactivating with pepsin-treated (Fab) antibodies to albumin; mixture with bacterial cells (e.g., C. parvum), endotoxins or lipopolysaccharide components of Gram-negative bacteria; emulsion in physiologically acceptable oil vehicles (e.g., mannide monooleate (Aracel A)); or emulsion with a 20% solution of a perfluorocarbon (Fluosol-DA®) used as a block substitute may also be employed to produce an adjuvant effect.

In addition to adjuvants, it may be desirable to co-administer biologic response modifiers (BRM) to enhance immune responses. BRMs have been shown to upregulate T cell immunity or downregulate suppressor cell activity. Such BRMs include, but are not limited to, Cimetidine (CIM; 1200 mg/d) (Smith/Kline, Pa.); or low-dose Cyclophosphamide (CYP; 300 mg/m2) (Johnson/Mead, N.J.) and cytokines such as 7-interferon, IL-2, or IL-12 or genes encoding proteins involved in immune helper functions, such as B-7.

### III. Examples

The following examples as well as the figures are included to demonstrate preferred embodiments of the invention. It should be appreciated by those of skill in the art that the techniques disclosed in the examples or figures represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Synthesis of the Neoglycoconjugate

The synthetic strategy for all three oligosaccharides G27_{SH}, G30_{SH}, and G28_{SH} relied on (a) the stereoselective α-galactosylation using Kiso's 4,6-di-tert-butylsilylene-galactosyl trichloroacetimidate donor; (b) an orthogonal protecting group strategy primarily based on acyl, acetal, and silyl groups; and (c) the use of mercaptopropyl glycosides for the conjugation to maleimide-derivatized BSA. The synthesis of GIPL-3-derived trisaccharide G28_{SH} also included a regioselective ring opening of the 4,6-O-di-tert-butylsilyene (DTBS) moiety to gain access to a disaccharide acceptor in a single step. The synthesis of the three target oligosaccharides G27_{SH}, G30_{SH}, and G28_{SH} started with the preparation of the monosaccharide building blocks 1, 2, and 3 (FIG. 3).

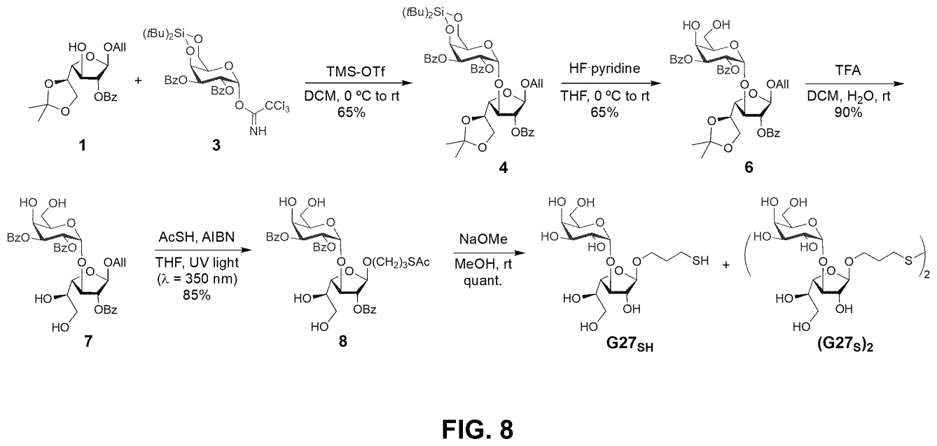

Synthesis of NGP27b. The monosaccharide building blocks 1-3 were synthesized according to known methods. Galβf acceptor 1 was glycosylated with Galαp donor 3 to furnish the fully protected disaccharide 4 in 65% yield (FIG. 8). The silylene group was removed using HF pyridine complex to give compound 6 in 65% yield, and then the isopropylidene group was hydrolyzed with aqueous trifluoroacetic acid (TFA) to afford allyl glycoside 7 in 90% yield. Radical addition of thioacetic acid (AcSH) to 7 in dry THF furnished thioester 8 in 85% yield (FIG. 8). Complete deacylation under Zemplen conditions provided the target disaccharide G27_{SH} quantitatively (FIG. 3), which oxidized to disulfide (G27s)₂ (FIG. 4). Upon reduction with TCEP.HCl and conjugation to commercial maleimide-derivatized BSA 14, NGP27b was obtained (FIG. 4).

Synthesis of NGP30b. The trisaccharide thiopropyl α-D-galactopyranosyl-(1→3)-β-D-galactofuranosyl-(1→3)-β-D-mannopyranoside G30_{SH} was synthesized by two consecutive glycosylation steps. First, the Galβf acceptor 2 was glycosylated with Galαp donor 3 with high stereoselectivity to produce disaccharide 5 in 67% yield (FIG. 3). This donor was used to regioselectively glycosylate mannosyl acceptor 9 to yield trisaccharide 10 in 47% yield (FIG. 3). Afterwards, the silylene protecting group was removed with HF pyridine complex, and without purification, both, the benzylidene and isopropylidene groups were removed by acid catalyzed hydrolysis to give compound 7 in 48% yield over two steps (FIG. 8). Radical addition of AcSH to the allyl glycoside was achieved in 85% yield to provide thioester 8 (FIG. 8). Finally, global removal of all ester groups under Zemplen conditions furnished the desired trisaccharide G30_{SH} quantitatively (FIG. 3), which oxidized to disulfide (G30s)₂ (FIG. 4). Upon reduction with TCEP HCl and conjugation to commercial maleimide-derivatized BSA 14, NGP30b was obtained (FIG. 4).

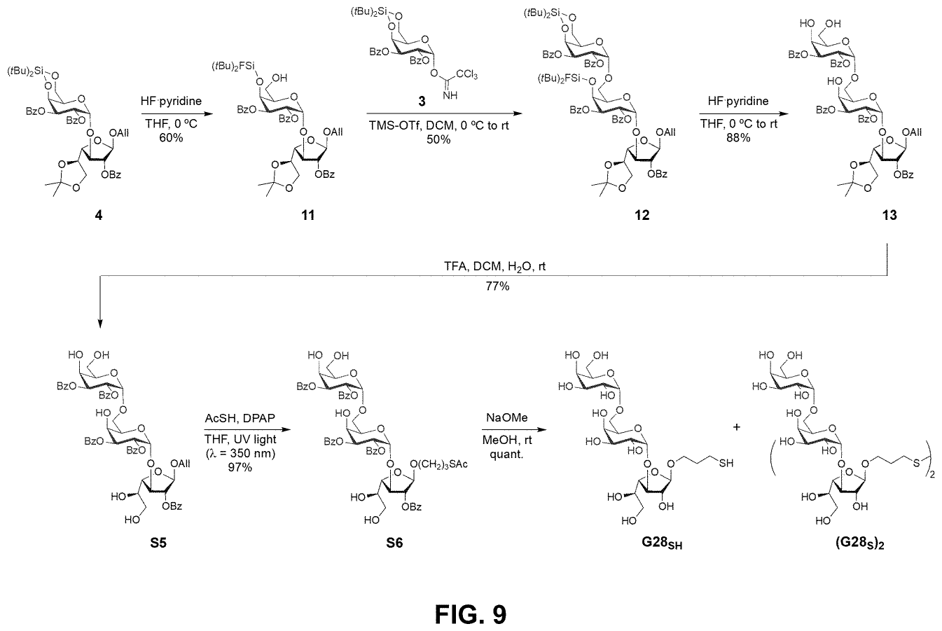

Synthesis of NGP28b. The trisaccharide mercaptopropyl α-D-galactopyranosyl-(1→6)-α-D-galactopyranosyl-(1→3)-β-D-galactofuranoside (G28_{SH}) was synthesized from disaccharide 4, which needed to be converted into a suitable acceptor. A regioselective ring opening of the 4,6-O-DTBS group was performed. Based on the known regioselective ring opening of a 3,5-O-DTBS group of a galactofuranoside, this should be accomplishable with tetrabutylammonium fluoride (TBAF). However, the 4,6-O-DTBS group of a galactopyranoside resists reaction via TBAF, even when applied in large access. A novel regioselective ring opening of the 4,6-O-DTBS group can be accomplished with HF pyridine complex at 0° C. Conversely, 1.2 equiv. of HF pyridine complex at 0° C. opened the silylene ring of disaccharide 4 within 6 h. The reaction time could be reduced to only 2 h by using HF pyridine in excess at 0° C. and immediate quenching with aqueous saturated NaHCO₃ solution. The resulting disaccharide 11 was obtained in 60% yield, and the inventors envisioned it to be used as a glycosyl acceptor (FIG. 9). One concern was that the bulky silyl group at position 4 could potentially hinder the glycosylation at OH-6, however, donor 3 was able to glycosylate acceptor 11 to provide the fully protected trisaccharide 12, with an exclusive α-stereoselectivity in an acceptable yield of 50%. Subsequent treatment with HF pyridine complex in excess from 0° C. to rt removed the silyl and silylene groups to produce trisaccharide 13 in 88% yield (FIG. 9). Acid-catalyzed hydrolysis of the isopropylidene group of 13 produced compound S5 in 77% yield, and the thioester derivative S6 was prepared by radical addition of AcSH in 93% yield (FIG. 9). Global deprotection under Zemplen conditions furnished the target mercaptopropyl trisaccharide G28_{SH} quantitatively (FIG. 3), which oxidized to disulfide 17. Reduction and conjugation to maleimide derivatized BSA 18 afforded NGP28b (FIG. 4).

### Example 2

Antigens NGP27b, NGP30b, and NGP28b Binds with IgG Anti-α-Gal Antibodies from CL Patient Sera with Acute L. major Infection

With the NGP antigens NGP27b, NGP30b, and NGP28b, IgG antibody responses of CL patient sera with acute L. major or L. fropica infection, confirmed by PCR, was studied by chemiluminescent ELISA. The patient sera had been collected from 120 individuals, mostly from the areas Al Ahsa and Asir in Saudi Arabia, where L. major and L. tropica, respectively, are endemic. In addition, pooled sera of 10 healthy individuals from England, United Kingdom, designated normal human serum (NHS), from Al Ahsa and Asir governorates. Additionally, 24 sera from individuals with skin pathologies (14 with eczema, 2 with bacterial infection, 2 with fungal infection, and 6 with unavailable diagnosis) that could confounded as CL, from Saudi Arabia (21 from Al Ahsa governorate and 3 from unknown provenance), were also integrated in the study. The heterologous sera are from patients who have skin conditions other than CL. Therefore, these sera represent a real-life control group, relevant to what a physician may encounter, from which a useful CL biomarker must be able to distinguish. Since all healthy individuals contain the natural (or NHS) anti-α-Gal antibodies directed against α-Gal containing antigens of enterobacteria (Galili et al., J. Exp. Med. 1984, 160, 1519-1531), a small amount of crossreactivity between NHS anti-α-Gal antibodies and the L. major-derived glycostructures is expected due to nonspecific binding. To ensure that ELISA responses were not a result of antibody binding to BSA or the crosslinker, 2-ME-BSA (also known as 2-MEb), obtained by conjugating 2-mercaptoethanol (2-ME) to maleimide-derivatized BSA 14 (FIG. 4), was used as a negative control antigen.

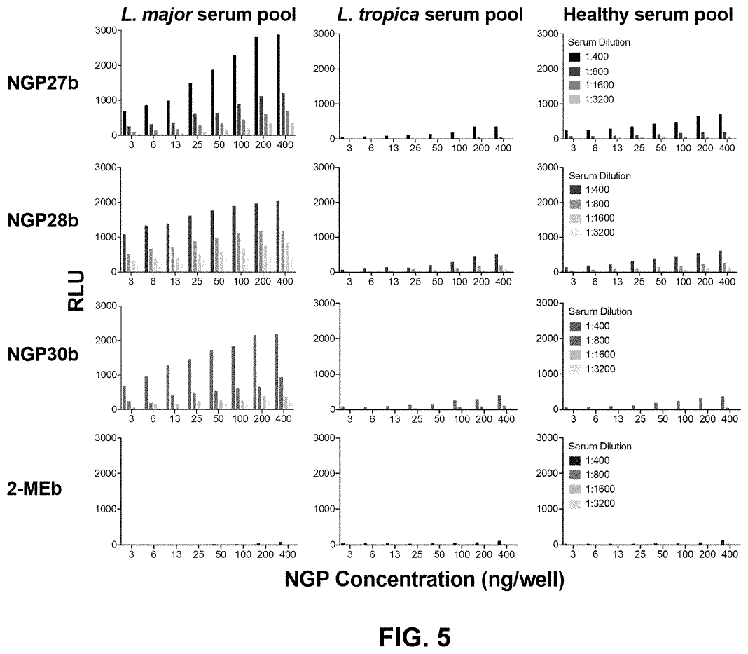

To identify a suitable protocol for the chemiluminescent ELISA, sera pools were prepared, and sera dilutions were cross-titrated against antigen quantity [ng/well]. FIG. 5 shows that the pooled sera of patients with an active L. major infections have a strong antibody response to NGP27b, NGP28b, and NGP30b, while the pooled sera of L. fropica and NHS showed significantly less antibody reactivity. The cross-titration experiment revealed that a serum dilution of 1:800 and an antigen loading of 25 ng/well shows excellent differential antibody reactivities of L. major positive sera vs. L. fropica positive sera or NHS. Under these conditions, the reactivity ratios between L. major and NHS serum pools were ˜6-, ˜9-, and ˜56-fold for NGP27b, NGP28b, and NGP30b, respectively. Comparing L. major vs. L. fropica serum pools, the reactivity ratios were ˜90-, ˜9-, and ˜16-fold for NGP27b, NGP28b, and NGP30b, respectively. As expected, the negative control 2-MEb showed essentially very little or no reactivity with all pooled sera, indicating that no significant antibody binding occurred to BSA or the crosslinker.

TABLE 1
Reactivity of L. major, L. tropica and heterologous sera with NGP27b, NGP28b, or
NGP30b in chemiluminescent ELISA.

| | | NGP27b | | NGP28b | | NGP30b | |
|---|---|---|---|---|---|---|---|
| Infection/Disease | n | Positive | Negative | Positive | Negative | Positive | Negative |
| Original values ^{a} | | | | | | | |
| L. major | 81 | 77 | 4 | 70 | 11 | 73 | 8 |
| Heterologous | 24 | 5 | 19 | 6 | 18 | 6 | 18 |
| L. tropica | 15 | 1 | 14 | 2 | 13 | 4 | 11 |
| Post-TG-ROC analysis ^{b} | | | | | | | |
| L. major | 81 | 76 | 5 | 65 | 16 | 70 | 11 |
| Heterologous | 24 | 4 | 20 | 7 | 17 | 0 | 24 |
| L. tropica | 15 | 0 | 15 | 3 | 12 | 1 | 14 |

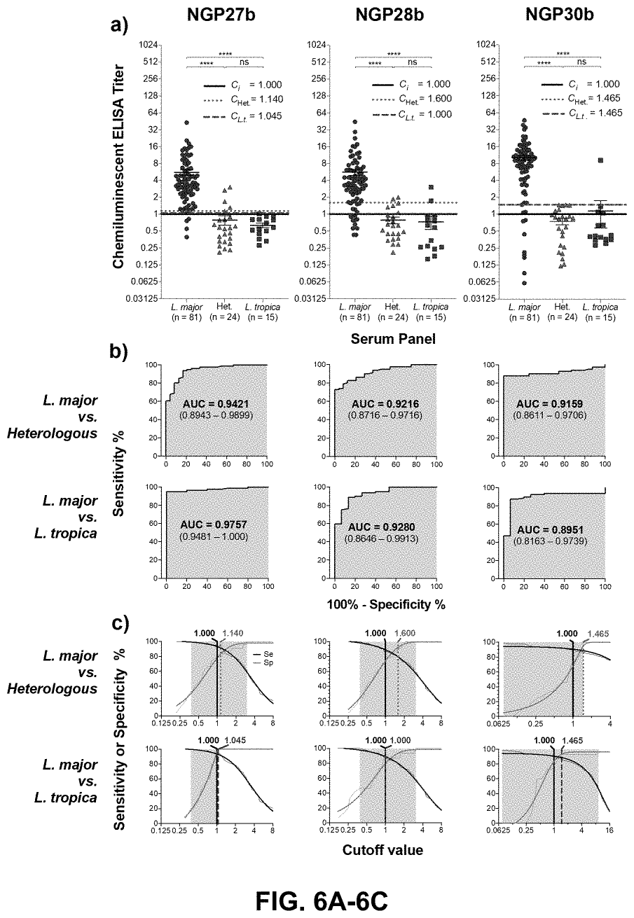

^{a} Values calculated based on the initial cutoff value (Cᵢ; titer
^{b} Values calculated based on the TG-ROC analysis (FIG. 6).

Next, all NGPs (NGP27b, NGP28b, and NGP30b) were put to a test as potential BMKs for L. major infections, and in particular differentiating these from heterologous diseases, which is a major issue in clinical settings in some endemic and nonendemic regions, where there is a high migration of CL patients from affected areas. Second, these NGPs were evaluated for their utility for distinguishing L. major from L. tropica infections, which is another challenge in similar clinical settings. To that end, the three NGPs were assessed by chemiluminescent ELISA, using conditions previously established (FIG. 5). Individual sera were assayed from CL patients chronically infected with L. major (n=81) or L. fropica (n=15), or patients with heterologous diseases (n=24).

Initially a chemiluminescent ELISA titer cutoff of 1.000 was used, which was determined in each immunoassay microplate by using a pool of negative control sera (healthy individuals from the UK, n=10), in duplicate or triplicate. Data showed that NGP27b diagnosed as positive 77/81 (sensitivity=95.3%) of the sera from patients with L. major infection, previously confirmed by dermatological examination and laboratory assays (lesion aspirate microscopy and ITS1-PCR-RFLP analysis) (Owino et al., PLoS Negl Trop Dis 2019, 13, e0007712) (FIG. 6, Table 1). On the other hand, NGP28b and NGP30b diagnosed as L. major-positive 70/81 and 73/81 (sensitivity=88.0% and 91.0%), respectively (FIG. 6, Table 1).

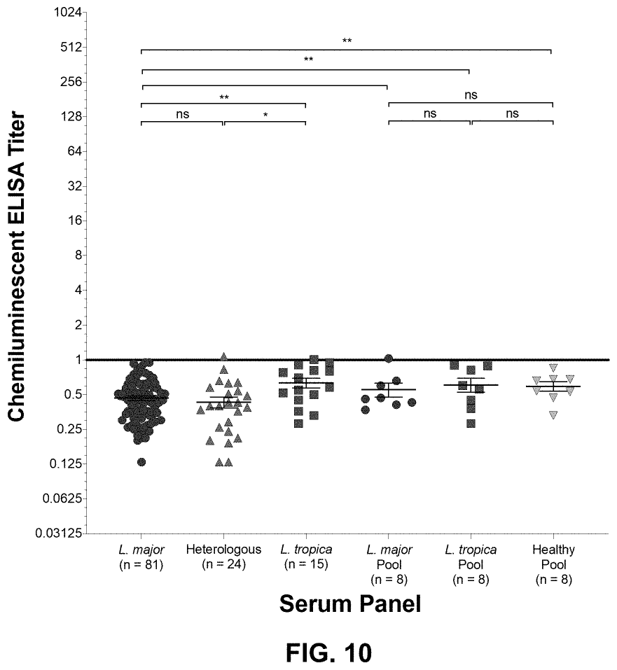

The three NGPs were also evaluated for specificity by comparing sera from L. major infections with sera from heterologous diseases or L. fropica infections. When assessed L. major-positive sera vs. sera from heterologous diseases, NGP27b, NGP28b, and NGP30b exhibited a specificity of 82.8, 80.0, and 80.0%, respectively (TABLE 2). When compared L. major-positive vs. L. fropica-positive sera, it was found that NGP27b, NGP28b, and NGP30b showed a specificity of 93.8, 88.2, and 79.0%, respectively (TABLE 2). Conversely, individual or pooled sera from L. major or L. fropica infections, or heterologous diseases showed very weak reactivity (mostly below the titer cutoff of 1.000) with the negative control antigen (2-MEb), strongly indicating that the antibody reactivity of all tested sera to the linker or the BSA carrier protein was negligible (FIG. 10).

TABLE 2
Sensitivity, specificity, and other diagnostic parameters of NGP27b, NGP28b, and NGP30b
in chemiluminescent ELISA.

| | L. major infections vs. | | | | | |
|---|---|---|---|---|---|---|
| | heterologous diseases | | | L. major vs. L. tropica infections | | |
| | NG27b | NGP28b | NGP30b | NG27b | NGP28b | NGP30b |
| | (%) | (%) | (%) | (%) | (%) | (%) |
| Original values ^{a} | | | | | | |
| Sensitivity ^{b} | 95.3 | 88.0 | 91.0 | 95.3 | 88.0 | 91.0 |
| Specificity ^{c} | 82.8 | 80.0 | 80.0 | 93.8 | 88.2 | 79.0 |
| FPR ^{d} | 17.2 | 20.0 | 20.0 | 6.2 | 11.8 | 21.0 |
| PPV ^{e} | 94.2 | 93.1 | 93.1 | 98.8 | 97.6 | 95.3 |
| NPV ^{f} | 85.7 | 68.6 | 75.0 | 79.0 | 57.7 | 65.2 |
| Post-TG-ROC analysis ^{g} | | | | | | |
| Sensitivity | 94.2 | 83.5 | 88.0 | 95.3 | 88.0 | 88.0 |
| Specificity | 85.7 | 92.3 | 100.0 | 100.0 | 88.2 | 93.8 |
| FPR | 14.3 | 7.7 | 0.0 | 0.0 | 11.8 | 6.2 |
| PPV | 95.3 | 92.0 | 100.0 | 100.0 | 96.4 | 98.8 |
| NPV | 82.8 | 60.0 | 75.0 | 75.0 | 48.4 | 57.7 |

^{a} Values calculated based on the initial cutoff value (Cᵢ; titer = 1.000).
^{b} Sensitivity = True Positive (TP)/TP + False Negative (FN)
^{c} Specificity = True Negative (TN)/TN + False Positive (FP)
^{d} False-positive rate = 100 − Specificity
^{e} Positive predictive value = TP/TP + FP
^{f} Negative predictive value = TN/TN + FN
^{g} Values calculated based on the TG-ROC analysis (FIG. 6c).

To compare the usefulness of the three NGPs for correctly discriminating true-positive (TP) from false-positive (FP) results, at various threshold (cutoff) values, receiver operating characteristic (ROC) curves were plotted (FIG. 6b). The area under the curve (AUC) values of the ROC curves for NGP27b (0.9421), NGP28b (0.9216), and NGP30b (0.9159), in the comparison of serum samples from L. major infections vs. heterologous diseases, indicated that NGP27b exhibited higher sensitivity and specificity than NGP28b and NGP30b (FIG. 6b, top graphs; Table 2). In the comparison of serum samples from L. major vs. L. tropica infections, the AUC values for NGP27b (0.9757), NGP28b (0.9280), and NGP30b (0.8951), indicated the same trend (FIG. 6b, bottom graphs; TABLE 2). Taken together, the data indicated that NGP27b showed a higher sensitivity and specificity than NGP28b and NGP30b.

Next, to fine-tune the initial titer cutoff value (Cᵢ) (FIG. 6a) for each NGP, a TG-ROC analysis was performed by plotting the ROC data (FIG. 6b) for sensitivity (Se) and specificity (Sp) as a function of the cutoff value, as described by Greiner et al. (Prev Vet Med 2000, 45, 23-41) (FIG. 6C). The selection of the cutoff value is always a trade-off between sensitivity and specificity, and it depends on the context in which the diagnostic BMK is to be applied. Since CL caused by L. major or L. tropica in endemic countries like Saudi Arabia, where little geographical overlap of the two infections exists, the utility of a new diagnostic BMK test is not a high priority. However, in areas where L. major and L. tropica infections coexist, for instance in conflict-affected countries (e.g., Afghanistan, Syria, Lebanon) (El Safadi et al., Trans R Soc Trop Med Hyg 2019, 113, 471-476; Ozaras et al., Expert Rev Anti Infect Ther 2016, 14, 547-55), and in nonendemic regions (e.g., Europe) with high migration from affected areas (Ready, Euro Surveill 2010, 15, 19505; Torpiano and Pace, Expert Rev Anti Infect Ther 2015, 13, 1123-38), there is an urgent need for new diagnostic BMKs that could accurately diagnose CL from non-CL conditions (i.e., dermatological diseases), and discriminate CL caused by different Leishmania species. In this context, a high specificity is preferred over high sensitivity for any potential new diagnostic BMK for CL (Greiner et al., Prev Vet Med 2000, 45, 23-41). When comparing L. major infections vs. heterologous diseases, an adjusted titer cutoff value for NGP27b of 1.140 (instead of 1.000) slightly decreased sensitivity to 94.2% (from 95.3%), but increased specificity to 85.7% (from 82.8%) (TABLE 2, Post-TG-ROC analysis). When comparing L. major vs. L. tropica infections, it was noticed that an adjusted titer cutoff value for NGP27b of 1.045 afforded a perfect specificity of 100% (from 93.8%), while maintaining the same high sensitivity of 95.3% (FIG. 6A,C; TABLE 2). For NGP28b, in the comparison of L. major infections vs. heterologous diseases, an adjusted titer cutoff value of 1.600 gave a lower sensitivity of 83.5% (from 88.0⁰/), while the specificity considerably improved to 92.3% (from 80.0%). Nevertheless, when comparing L. major vs. L. tropica infections, it was found that the original NGP28b titer cutoff value of 1.000 could not be significantly improved without drastically affecting the sensitivity. Therefore, the same cutoff value of 1.000 was maintained (FIG. 6A,C; TABLE 2). Finally, for NGP30b, when compared L. major infections vs. heterologous diseases, an adjusted titer cutoff value of 1.465 gave a sensitivity of 88% (from 91%), but significantly increased the specificity to 100% (from 80.0%) (FIG. 6A,C; TABLE 2). When comparing L. major vs. L. tropica infections with NGP30b, the same adjusted titer cutoff value of 1.465 decreased the sensitivity to 88% (from 91.0%), but significantly increased the specificity to 93.8% (from 79.0%).

Based on the TG-ROC analysis data with the adjusted titer cutoff values, an algorithm can be proposed with the two NGPs that exhibited the best outcomes in terms of sensitivity and specificity, i.e., NGP27b and NGP30b, to consecutively screen sera from patients who could be infected with either L. major or L. tropica, or affected with confounding, non-CL dermatological condition(s) (FIG. 7). First, the serum would be screened by chemiluminescent ELISA with NGP27b. A positive result would indicate L. major infection or heterologous disease, whereas a negative result would indicate L. tropica infection or heterologous disease. A sample with a positive result would undergo a second chemiluminescent ELISA now using NGP30b to discriminate between L. major infection and heterologous disease. Importantly, the proposed algorithm should complement the patient's clinical assessment and history.

Although all three NGPs exhibited similar trends regarding their immunoreactivity with L. major sera, on average NGP30b showed titers about twice as high as those of NGP27b and NGP28b. This could be explained by the fact that NGP30b contains a larger portion of the glycotope of type-II GIPL-2, which has been shown to be strongly recognized by sera from L. major patients (McConville et al., J Biol Chem 1990, 265, 7385-94).

Regardless the NGP tested, the inventors observed a small cross-reactivity with sera from L. tropica infections and heterologous diseases. A plausible explanation for such a cross-reactivity could be the presence of natural anti-α-Gal antibodies, abundantly present in the serum of all individuals, as reported by Galili et al. (Immunology 2013, 140, 1-11; Macher and Galili, Biochim Biophys Acta 2008, 1780, 75-88). These antibodies cross-react with the so called Galili epitope or trisaccharide (Galα1,3Galβ1,4GlcNAc-R) and other glycans containing terminal, nonreducing α-Gal epitopes, in particular melibiose (Galα1,6Glc) (Almeida et al., J Immunol 1991, 146, 2394-400; Almeida et al., Biochem J 1994, 304 (Pt 3), 793-802; Ashmus et al., Org Biomol Chem 2013, 11, 5579-83; Schocker et al., Coupling and Decoupling of Diverse Molecular Units in Glycosciences 2018, 195-211; Schocker et al., Glycobiology 2016, 26, 39-50; Subramaniam et al., Parasitology 2018, 145, 1758-1764). In fact, both the Galili trisaccharide and melibiose are regularly used for the purification of natural anti-α-Gal antibodies (Almeida et al., J Immunol 1991, 146, 2394-400; Galili et al., J Ep Med 1984, 160, 1519-31), which could explain at least in part the cross-reactivity observed here with NGP27b (Galα1,3Galfβ-BSA) and NGP30b (Galα1,3Galfβ1,3Manα-BSA), and NGP28b (Galα1,6Galα1,3Galfβ-BSA), which contain terminal α-Gal residue with the same linkages as observed in the Galili trisaccharide and melibiose.

The chemical synthesis of α-Gal-containing oligosaccharides (G27_{SH}, G30_{SH}, and G28_{SH}) and their immunological evaluation with sera from OWCL patients enabled the identification of diagnostic BMKs for distinguishing OWCL caused by L. major from heterologous diseases, and from L. fropica infections. The synthetic targets were the mercaptopropyl glycosides of Galpα1,3Galβ (G27_{SH}), Galpα1,3Galfβ1,3Manα (G30_{SH}), and Galpα1,6Galpα1,3Galfβ (G28_{SH}) equipped with a handle for conjugation. These oligosaccharides correspond to the terminal di- and trisaccharide moieties of type-II GIPL-2, and the terminal trisaccharide of GIPL-3, respectively, which are abundantly expressed in L. major, but absent (or much less abundant) in L. fropica. The 4,6-O-DTBS protecting group of Galp played two important roles in their synthesis: (a) it allowed for stereoselective α-galactosylation; and (b) it underwent a regioselective ring-opening reaction producing a new Gal acceptor in a single step, which allowed for a convenient synthesis of G28_{SH}. Conjugation of the saccharides G27_{SH}, G28_{SH}, and G30_{SH} to maleimide-derivatized BSA produced NGPs antigens for chemiluminescent ELISA. The two neoglycoproteins, NGP27b and NGP30b, both derived from L. major GIPL-2, exhibited 100% specificity for the distinction of L. major from L. fropica infections (NGP27b), and heterologous diseases (NGP30b). Therefore, sera of patients with skin lesions that are suspicious for OWCL can be subjected to two consecutive or parallel chemiluminescent ELISA tests, which will diagnose an L. major infection with a very high level of confidence. These NGPs could potentially be used to develop a species-specific lateral flow test for OWCL, which is important for informing best treatment options. This is especially relevant in areas of population displacement in the Middle East with large numbers of refugees who migrated from OWCL-endemic areas.

### Example 3

### Serodiagnosis and Therapeutic Monitoring of New-World Tegumentary Leishmaniasis Using Synthetic Type-2 Glycoinositolphospholipid-Based Neoglycoproteins

A. Results

This cross-sectional, retrospective study evaluated a cohort of patients with distinct clinical forms of TL caused by L. braziliensis, and individuals with the asymptomatic subclinical (SC) form of the infection. These patients are from the region of Corte de Pedra, Bahia, Brazil, which is a well-studied endemic area for TL (Jirmanus et al., Am J Trop Med Hyg. 2012 86(3):426-33). Table 3 shows the demographics and clinical characteristics of the 80 individuals tested across the TL spectrum: the mean age ranged from 26 to 45 years (33.8 f 16.2), with a predominance of males (62.5%). SC individuals were significantly younger (26 f 13.9 years old) than ML (45 f 18) and DL (39 f 15) patients (p=0.0002, ML vs SC; p=0.006, DL vs SC). Most CL patients (82.4%) exhibited a single lesion, whereas DL patients exhibited a median of 20 ulcers (Table 3). For CL and ML patients, clinical cure was defined by complete healing of the ulcers with reepithelialization without raised borders on day 180 after initiation of treatment (Aronson et al., Clin Infect Dis. 2016 63(12):e202-e264).

TABLE 3
Demographics and clinical characteristics of tegumentary leishmaniasis patients.

| | TL form ^{a} | | | | |
|---|---|---|---|---|---|
| Variable | CL (n = 17) | ML (n = 16) | DL (n = 16) | SC (n = 31) | p ^{b} |
| Age. years ^{c} | 32 (12.4) | 45 (18) | 39 (15) ^{e} | 26 (13.9) | 0.02 (CL vs. ML): 0.0002 (ML vs. SC); |
| | | | | | 0.006 (DL vs. SC) |
| Number of males | 12 (70.6) | 7 (43.8) | 15(93.8) | 16 (51.6) | — |
| (%) | | | | | |
| Number of lesions ^{d} | 1 (1) | — | 20 (13-35) ^{e} | — | — |
| LST area (mm²) ^{a,d} | 204 | 266 (147.5-319)^{f} | 113 | 104 | <0.0001 (CL vs. DL); 0.0067 (ML |
| | (170.3-261.4) | | (4.8-175.8) | (44.0-240.2) | vs.DL); 0.0380 (CL vs. SC); 0.0299 |
| | | | | | (ML vs. SC) |
| Healing time | 45.1 (18.6) | 59.6 (17.7) | 137.8 (61.9) ^{e} | — | 0.0294 (CL vs. ML); 0.0001 (CL vs. |
| (days) ^{e} | | | | | DL); 0.0001 (ML vs. DL) |

^{a} Abbreviations: LST, leishmanin (Montenegro) skin test; CL, cutaneous leishmaniasis; ML, mucocutaneous leishmaniasis; DL, disseminated leishmamasis; SC, subelinical leishmaniasis.
^{b} The Student's t- test or Mann Whitney test were used to compare continuous variables and the Fisher's exact test to compare proportions.
^{c} Mean(SD)
^{d} Median (interquartile range, IQR)
^{e} Data missing from one subject.
^{f} Data missing from three subjects.

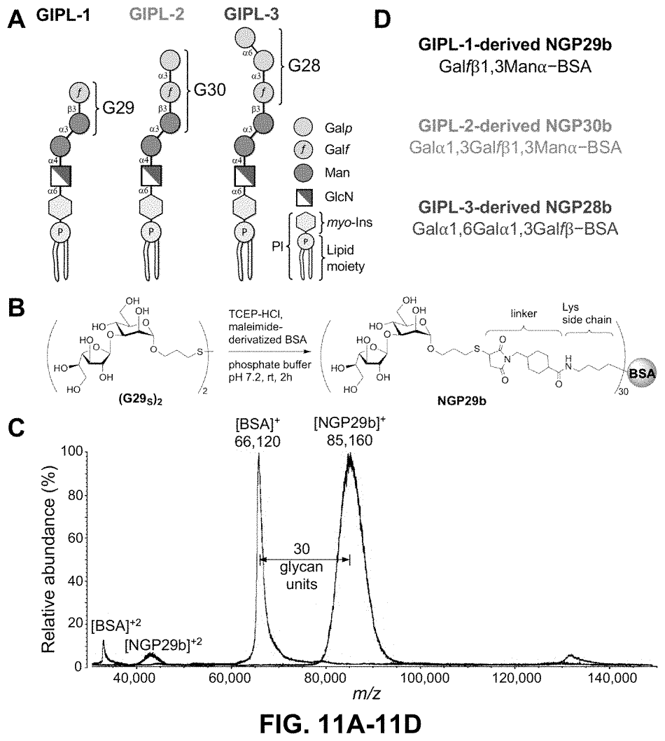

For the evaluation of the presence of L. braziliensis-specific anti-α-Gal and anti-β-Galf antibodies in the sera of CL, ML, DL, and SC patients, three synthetic NGPs containing terminal glycotopes found on L. major type-2 GIPL-1, -2, and -3 were employed, these NGPs have previously shown to be highly reactive to sera from American TL caused by L. braziliensis (Avila and Rojas, J Clin Microbiol. 1990 28(7):1530-7). In FIG. 11, the schematic representations of L. major type-2 GIPL-1, -2, and -3, the chemical synthesis, representative quality control (by MALDI-TOF-MS), and the basic composition of NGP29b, NGP30b, and NGP28b are shown. The inclusion of the terminal, nonreducing β-Galf-bearing NGP29b in this study was based on the premise that β-Galf is a sugar entirely absent in all mammals, thus extremely immunogenic, immunodulatory, and antigenic to mice and/or humans, hence a potential BMK for American TL (Montoya et al., Molecules. 2022 27(2):411; de Lederkremer and Colli, Glycobiology. 1995 5(6):547-52; Travassos and Almeida, Springer Semin Immunopathol. 1993 15(2-3):183-204; Golgher et al., Mol Biochem Parasitol. 1993 60(2):249-64; Suzuki et al., Infect Immun. 2002 70(12):6592-6; Schnaidman et al., The Journal of protozoology. 1986 33(2):186-91).

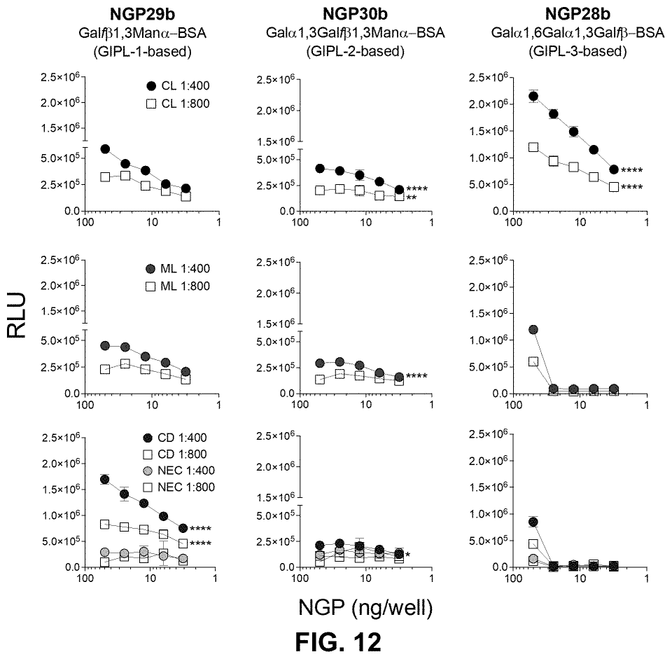

Next, the seroreactivity of CL, ML, CD patients and NECs to these three synthetic NGPs were tested. Pooled sera (n=15) from each patient/control panel were tested at 1:400 or 1:800 dilution against a concentration range (50-3.1 ng/well) of the three NGPs. The serum pools from CL or ML patients exhibited discrete reactivity to NGP29b (GIPL-1-based), whereas a serum pool from CD patients showed strong reactivity to this NGP, in a dose-dependent manner, at 1:400 and 1:800 dilutions (FIG. 12, left panels). NGP30b (GIPL-2-based) was detected with similar RLU value by serum pools from ML and CD patients, and NEC individuals, with a slightly increase in recognition by the serum pool from CL patients at 1:400 dilution (FIG. 12, central panels). Conversely, the serum pool from CL patients exhibited a strong reactivity to NGP28b (GIPL-3-based), at both dilutions tested, in a dose-response manner, whereas serum pools from ML and CD patients exhibited reactivity comparable to NEC individuals at concentrations lower than 25 ng/well. Serum pools from NEC showed very weak reactivity to NGP28b at all concentrations tested (FIG. 12, right panels). Taken together, these results indicate that the GIPL-3-based NGP28b is strongly recognized by anti-α-Gal antibodies present in the serum pool from L. braziliensis-caused CL, and to a much lesser extent by ML and CD serum pools. By contrast, GIPL-1-based NGP29b is more strongly reactive to anti-β-Galf antibodies present in the serum pool from CD patients than those in CL and ML serum pools.

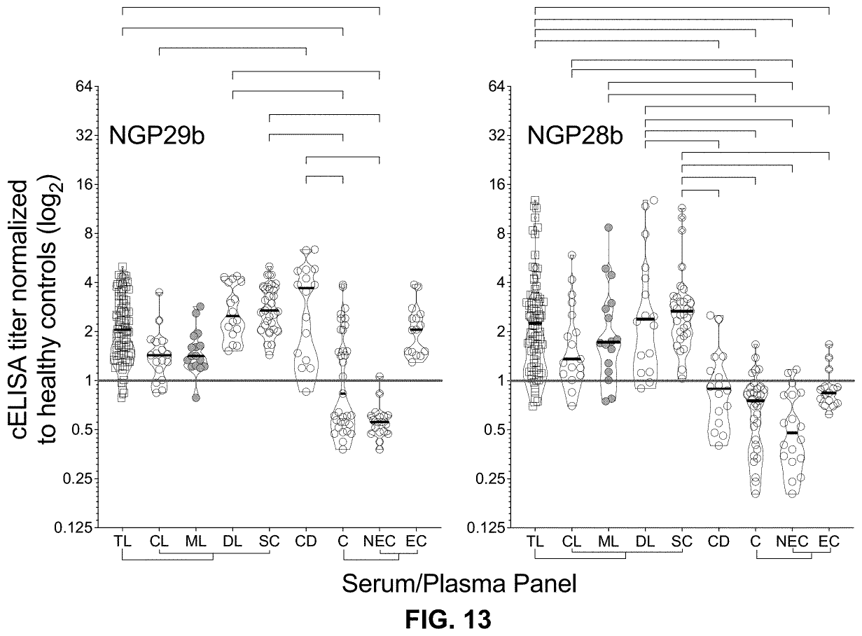

This initial serological survey was expanded to individual patients representing the full TL clinical spectrum, CD patients, and endemic (non-TL) and nonendemic healthy controls. Despite the diversity in clinical presentations, sera from patients across all clinical forms of TL reacted strongly to NGP29b and NGP28b (FIG. 13). An initial cELISA titer cutoff (Cᵢ) of 1.000 was established, determined in each microplate assay by using a pool of seemingly healthy nonendemic control sera (NEC, n=15) in sextuplicate, as described in Material and Methods. NGP29b diagnosed 76/80 (sensitivity=95.0%) of all TL patients as positive, being 14/17 (sensitivity=82.4%) of CL, 15/16 (sensitivity=93.8%) of ML, and 16/16 (sensitivity=100%) of DL patients. (FIG. 13, left panel; Table 4 and Table 6). On the other hand, NGP28b diagnosed as positive 74/80 (sensitivity=92.5%) of total TL patients, being 15/17 (sensitivity=88.2%) of CL patients, and 14/16 (sensitivity=87.5%) of both ML and DL patients. For CD patients, NGP29b exhibited a 93.8% sensitivity (15/16), whereas NGP28b showed a 37.5% sensitivity value (6/16 individuals). In fact, CD patients and total controls (C=EC+NEC) exhibited nonsignificant difference in the titers of anti-NGP28b antibodies (FIG. 13, right panel; Table 5 and Table 6). Both antigens, however, recognized as positive 100% of SC individuals, who do not present active ulcers but exhibit a positive LST response, an indicative of exposure to Leishmania spp. (Follador et al., Clin Infect Dis. 2002 34(11):E54-8).

TABLE 4
Sensitivity, specificity, and other diagnostic parameters
of type-2 GIPL-1-based NGP29b.

| | TL Infection Forms vs. Endemic and | | | | | |
|---|---|---|---|---|---|---|
| | Nonendemic Controls ^{a} | | | | | |
| Parameter | TL | CL | ML | DL | SC | CD |
| | Original Values (%) ^{b} | | | | | |
| Sensitivity | 95.0 | 82.4 | 93.8 | 100.0 | 100.0 | 93.8 |
| Specificity | 48.5 | 48.5 | 48.5 | 48.5 | 48.5 | 48.5 |
| FPR | 51.5 | 51.5 | 51.5 | 51.5 | 51.5 | 51.5 |
| PPV | 80.0 | 45.2 | 46.9 | 48.5 | 64.6 | 46.9 |
| NPV | 81.4 | 84.2 | 94.1 | 100.0 | 100.0 | 94.1 |

^{a} Controls: endemic (EC) (n = 15) plus healthy nonendemic (NEC) (n = 18) control individuals.
^{b} Values calculated based on the initial cutoff value (Ci; titer = 1.000) (FIG. 3A), as described in Material and Methods. Sensitivity = true positive (TP)/TP + false negative (FN). Specificity = true negative (TN)/TN + false positive (FP). False-positive rate (FPR) = 100 − specificity. Positive predictive value (PPV) = TP/TP + FP. Negative predictive value (NPV) = TN/TN + FN.

TABLE 6
Seroreactivity to NGP29b (GIPL-1-based) and
NGP28b (GIPL-3-based) of different
clinical forms of tegumentary leishmaniasis.

| Disease/Clinical | | NGP29b | | NGP28b | |
|---|---|---|---|---|---|
| form/control | n | Positive | Negative | Positive | Negative |
| | Original Values^{a} | | | | |
| Tegumentary | 80 | 76 | 4 | 74 | 6 |
| leishmaniasis | | | | | |
| CL | 17 | 14 | 3 | 15 | 2 |
| ML | 16 | 15 | 1 | 14 | 2 |
| DL | 16 | 16 | 0 | 14 | 2 |
| SC | 31 | 31 | 0 | 31 | 0 |
| Chagas disease | 16 | 15 | 1 | 6 | 10 |
| Endemic control | 15 | 15 | 0 | 3 | 12 |
| | Post-TG-ROC Analysis Values^{b} | | | | |
| Tegumentary | 80 | 76 | 4 | 74 | 6 |
| leishmaniasis | | | | | |
| CL | 17 | 14 | 3 | 15 | 2 |
| ML | 16 | 15 | 1 | 14 | 2 |
| DL | 16 | 16 | 0 | 15 | 1 |
| SC | 31 | 31 | 0 | 29 | 2 |
| Chagas disease | 16 | 15 | 1 | 6 | 10 |
| Endemic control | 15 | 15 | 0 | 3 | 12 |

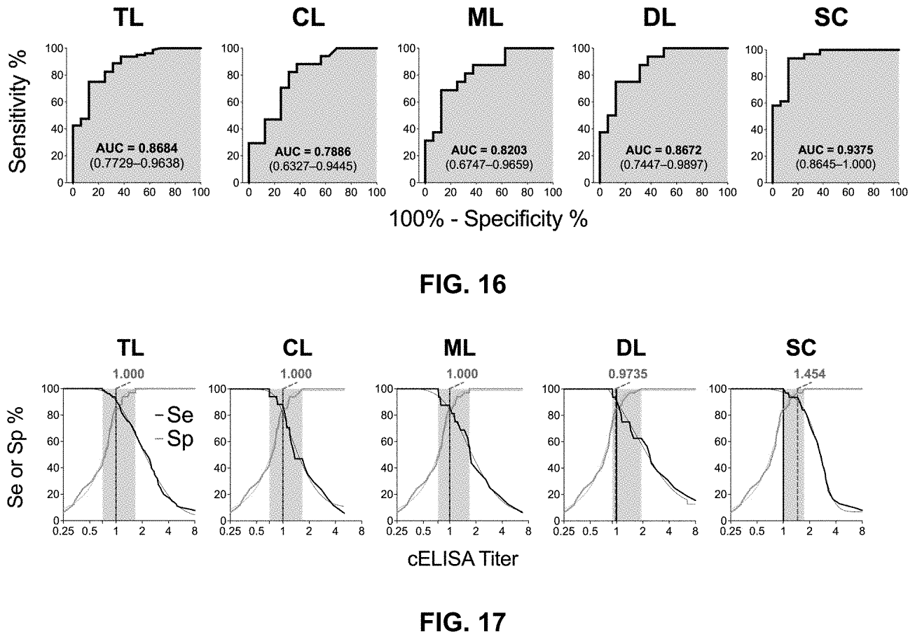

^{a}Values calculated based on the initial cutoff value (Ci; titer = 1.000) (FIG. 13), as described in Materials and Methods.
^{b}Values calculated based on the TG-ROC analysis(see FIG. 16 and Table 5).

NGPs have been evaluated for specificity by comparing sera from the TL forms caused by L. braziliensis with control (EC and NEC) sera. NGP29b exhibited a low specificity of 48.5% when sera from all TL clinical forms studied was evaluated and CD (Table 4). This result was due to 100% of EC sera (n=15) being diagnosed as false-positive by NGP29b (FIG. 13, left panel). Conversely, NGP28b exhibited 84.9% specificity comparing sera from total TL forms or individual CL, ML, DL, or SC form vs EC and NEC controls (Table 5). NGP28b also successfully discriminated patients of all TL clinical forms from CD patients, with 92.5% sensitivity, 62.5% specificity, and AUC=0.8684, indicating a strong discriminatory power (Table 7 and FIG. 16).

TABLE 5
Sensitivity, specificity, and other diagnostic parameters of type-2 GIPL-3-based NGP28b,
in the comparison of different TL clinical forms vs. endemic and nonendemic controls.

| | TL Clinical Forms vs. Endemic and Nonendemic Controls ^{a} | | | | | |
|---|---|---|---|---|---|---|
| Parameter | TL | CL | ML | DL | SC | CD |
| | Original values (%) ^{b} | | | | | |
| Sensitivity | 92.5 | 88.2 | 87.5 | 87.5 | 100.0 | 37.5 |
| Specificity | 84.9 | 84.9 | 84.9 | 84.9 | 84.9 | 84.9 |
| FPR | 15.2 | 15.2 | 15.2 | 15.2 | 15.2 | 15.2 |
| PPV | 93.7 | 75.0 | 73.7 | 73.7 | 86.1 | 54.6 |
| NPV | 82.4 | 93.3 | 93.3 | 93.3 | 100.0 | 73.7 |
| | Post-TG-ROC Analysis (%) ^{c} | | | | | |
| Sensitivity | 92.5 | 88.2 | 87.5 | 93.8 | 93.6 | 37.5 |
| Specificity | 84.9 | 84.9 | 84.9 | 84.9 | 97.0 | 84.9 |
| FPR | 15.2 | 15.2 | 15.2 | 15.2 | 3.0 | 15.2 |
| PPV | 93.7 | 75.0 | 73.7 | 75.0 | 85.3 | 54.6 |
| NPV | 82.4 | 93.3 | 93.3 | 96.6 | 93.3 | 73.7 |

^{a} Controls: endemic (EC) (n = 15) plus healthy nonendemic (NEC) (n = 18) individuals.
^{b} Values calculated based on the initial cutoff value (Ci; titer = 1.000) (FIG. 13B), as described in Material and Methods.
^{c} Values calculated based on the TG-ROC analysis (FIG. 17).

TABLE 7
Sensitivity, specificity, and other diagnostic parameters
of type-1 GIPL-3-based NGP28b, in comparison
of different TL clinical forms vs. Chagas disease.

| | TL Infection Forms vs Chagas Disease | | | | |
|---|---|---|---|---|---|
| | TL | CL | ML | DL | SC |
| Parameter | Original Values (%)^{a} | | | | |
| Sensitivity | 92.5 | 88.2 | 87.5 | 87.5 | 100.00 |
| Specificity | 62.5 | 62.5 | 62.5 | 62.5 | 62.5 |
| FPR | 37.5 | 37.5 | 37.5 | 37.5 | 37.5 |
| PPV | 92.5 | 71.4 | 70.0 | 70.0 | 83.8 |
| NPV | 92.5 | 83.3 | 83.3 | 83.3 | 100.0 |

^{a}Values calculated based on the initial cutoff value (Ci; titer = 1.000) (FIG. 13B), as described in Materials and Methods. Sensitivity = true positive (TP)/TP + false negative (FN). Specificity = true negative (TN)/TN = false positive (FP). False-positive rate (FPR) = 100 − specificity. Positive predictive value (PPV) = TP/TP + FP. Negative predictive value (NPV) = TN/TN + FN.

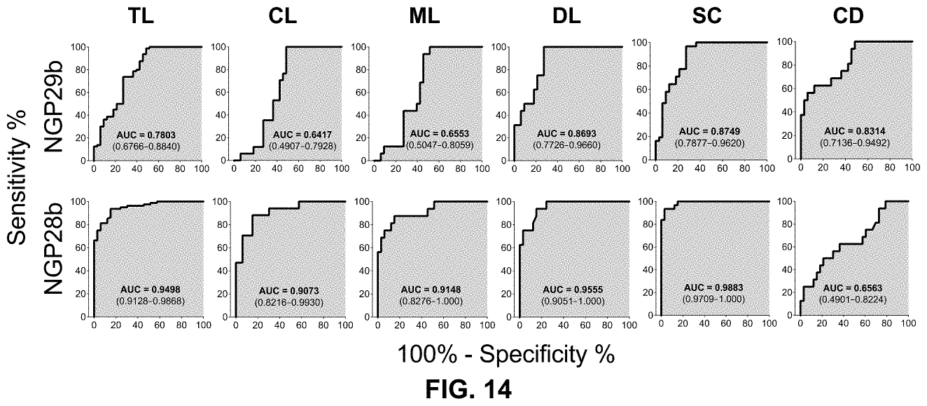

To further compare the capacity of NGP29b and NGP28b to discriminate sera from TL patients and SC individuals from EC and NEC sera, we performed ROC analysis using cELISA titers normalized to NEC serum pools. The AUC values of the ROC curves for the reactivity of NGP29b and NGP28b, respectively, with sera from total TL (0.7803 and 0.9498), CL (0.6417 and 0.9073), ML (0.6553 and 0.9148), DL (0.8693 and 0.9555), SC (0.8749 and 0.9883), and CD (0.8314 and 0.6563) patients confirmed the higher sensitivity and specificity of NGP28b compared to NGP29b across the different TL forms studied (FIG. 14).

There is an urgent need for new serological diagnostic BMKs that could detect the broad spectrum of clinical presentations in TL, especially for surveillance during pre-clinical phase or reactivation of disease. In this context, a high sensitivity is preferred over high specificity for a new potential biomarker for TL. To that end, we then performed a two-graph ROC (TG-ROC) analysis of NGP28b by plotting the ROC data for sensitivity (Se) and specificity (Sp) as a function of the cELISA titer that defines the original cutoff (Ci) value of 1.000, to fine-tune the analysis through cutoff adjustment (Greiner et al., Prev Vet Med. 2000, 45(1-2):23-41). For DL, the adjustment of the initial titer cutoff value (Cᵢ) of 1.000 to 0.9735 gave a higher sensitivity (93.8% from 87.5%), while maintaining the same specificity of 84.9%. For SC, although the adjustment from 1.000 to 1.454 of the titer cutoff value resulted in a lower sensitivity (93.6% from 100%), it significantly increased the specificity from 84.9% to 97%. The balance of cutoff values of sensitivity and specificity for total TL, CL, and ML diagnosis could not be significantly improved; therefore, we maintained the original Cᵢ of 1.000 (Table 3 and FIG. 17).

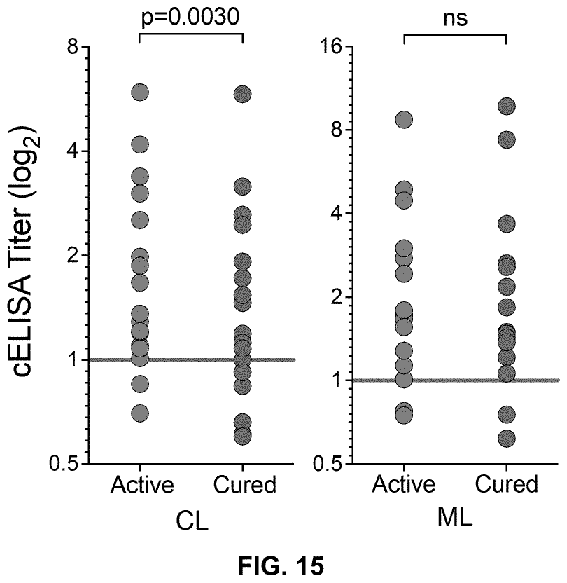

Titers of anti-Leishmania IgG antibodies are known to decrease after successful chemotherapy of CL and ML (Fagundes-Silva et al., Parasite Immunol. 2012, 34(10):486-91; de Lima et al., Front Cell Infect Microbiol. 2021, 11:652956). Possible explanations are related to decreased circulating antigen and/or modulation of the immune response following parasite elimination (Ribeiro-de-Jesus et al., Braz J Med Biol Res. 1998, 31(1):143-8). Therefore, the inventors also evaluated whether NGP28b-based cELISA could be used for monitoring cure of CL and ML patients, with matched samples of patients before treatment (active disease) and 90 days after the onset of treatment (cured). Sera from cured CL patients exhibited significantly lower titers of anti-NGP28b (p=0.003, Wilcoxon matched-pairs test) compared to serum samples from the same individuals with active disease. By contrast, overall, ML patients exhibited non-significant differences in the reactivity to NGP28b pre- and posttreatment (FIG. 15). Of note, CL patients reached clinical cure significantly faster (45.1±18.6 days) compared to ML patients (59.6±17.7 days; p=0.0294, CL vs. ML) (Table 3), which probably contributed for the lower titers of anti-NGP28b antibodies in the serum of cured CL patients (FIG. 15). Collectively, our results showed that serology to NGP28b, a L. major type-2 GIPL-3-based NGP is applicable for the serodiagnosis of different clinical forms of TL caused by L. braziliensis, specially of asymptomatic SC forms. Moreover, NGP28b-based cELISA has the potential to be used as a BMK to monitor clinical cure following chemotherapy in CL patients.

The cell surface of all Leishmania species thus far studied is covered by a dense coat of glycosylphosphatidylinositol (GPI)-anchored glycoconjugates, containing or not a polypeptide chain. Among those that lack protein, GIPLs and LPG are the most abundant and studied GPI-anchored glycoconjugates, particularly those from Old-World Leishmania species (e.g., L. major, L. donovani, L. tropica, L. aethiopica). We have recently shown that synthetic NGPs containing two similar α-Gal glycotopes, Galα1,3Galfβ-BSA (NGP27b) and Galα1,3Galfβ1,3Manα-BSA (NGP30b), based on L. major type-2 GIPL-3, were highly antigenic and able to discriminate Old-World CL caused by L. major from that caused by L. tropica (Montoya et al., JACS Au. 2021, 1(8):1275-1287). A previous study by Avila et al. has demonstrated that α-Gal-containing glycolipids purified from L. braziliensis promastigotes and comigrating with L. major type-2 GIPL-2 and GIPL-3 were highly antigenic for sera from patients with New-World or American TL caused by L. braziliensis (Avila et al., J Clin Microbiol. 1991, (10):2305-12). Thus far, the detailed structure of the L. braziliensis GIPLs remain elusive. However, a preliminary structural analysis by Assis et al. indicates that L. braziliensis GIPLs are rich in galactose residues and could be similar to type-2 GIPLs of L. major (Assis et al., PLoS Negl Trop Dis. 2012, 6(2):e1543), as previously proposed by Avila and colleagues (Avila et al., J Clin Microbiol. 1991, (10):2305-12). These studies made us to hypothesize that L. major type-2 GIPLs could be useful as diagnostic BMKs for different clinical forms of American TL.

As proof of concept, here we employed the reversed immunoglycomics approach (Montoya et al., JACS Au. 2021, 1(8):1275-1287), a bottom-up strategy that combines the chemical synthesis of potential glycotopes and conjugation to a carrier protein to generate NGPs, and probing them for antigenicity in serological immunoassays with patients' sera. To this end, using sera from patients with different clinical forms of TL caused by L. braziliensis, we evaluated one β-Galf-bearing NGP (NGP29b, Galfβ1,3Manα-BSA) and two α-Gal-NGPs (NGP30b, Galα1,3Galβ1,3Manα-BSA; and NGP28b, Galα1,6Galα1,3Galfβ-BSA), based on L. major type-2 GIPL-1, -2, and -3, respectively. α-Galp- and β-Galf-Containing glycotopes are abundant among trypanosomatids such as T. cruzi and some species of Leishmania, and are highly immunogenic to humans (Galili, In: Galili U, editor. The Natural Anti-Gal Antibody as Foe Turned Friend in Medicine. London, San Diego, Cambridge, Oxford: Academic Press, Elsevier, 2018, 57-71; de Lederkremer and Colli, Glycobiology. 1995, (6):547-52; Travassos and Almeida, Springer Semin Immunopathol. 1993, 15(2-3):183-204; Cabezas et al., Org Biomol Chem. 2015, (31):8393-404), making them potentially suitable for the purpose of specific and differential serodiagnosis of these diseases. Previous studies using New-World Leishmania species showed the potential of anti-α-Gal serological diagnosis in CL, ML, and diffuse cutaneous leishmaniasis (DCL) caused by L. Mexicana (Avila and Rojas, J Clin Microbiol. 1990, (7):1530-7), and in CL caused by L. braziliensis (de Souza et al., Parasitology. 2018, 145(14):1938-48; Avila and Rojas, J Clin Microbiol. 1990, (7):1530-7). Here, we showed by cELISA that type-2 GIPL-3-based NGP28b (Galα1,6Galα1,3Galfβ-BSA) was the most reactive NGP to CL sera, in a dose-dependent and specific manner, as well as with TL sera from patients with other clinical forms. Moreover, NGP28b exhibited low cross-reactivity to CD and EC sera, indicating a strong discriminatory power. On the other hand, although highly reactive to sera from the same TL cohort, NGP29b (Galfβ1,3Manα-BSA) was also highly cross-reactive to sera from CD patients and EC individuals, but not with sera from NEC individuals. This result entirely agrees with a recent study showing that CD patients have very high levels of anti-β-Galf IgG antibodies against Galfβ1,3Manα-BSA (NGP29b) and Galfβ1,3Manα1,2[Galfβ1,3]Manα-BSA (NGP32b) (Montoya et al., Molecules. 2022, 27(2):411). We cannot exclude the possibility of the EC individuals being infected by other infectious agents (e.g., fungi, bacteria, and/or parasites) that could elicit anti-β-Galf IgG antibodies that could strongly recognize NGP29b. The high immunoreactivity of NGP29b to CD and its lack of specificity for TL vs. EC represents an issue for the use of this NGP as a diagnostic tool in rural areas of Bahia, where T. cruzi vectors still occur in domestic and peridomestic environments (Mendonca et al., Am J Trop Med Hyg. 2015, 92(5):1076-80; Ribeiro et al., Parasit Vectors. 2019, 12(1):604). Thus, in areas where mixed CD and TL infections might occur, cross-reactivity to NGP29b could result in false-positive outcomes. Conversely, we showed that seroreactivity against NGP28b successfully discriminated TL from CD sera, with 92.5% sensitivity and 62.5% specificity (AUC=0.8684), indicating a strong discriminatory power of this antigen. Obviously, a higher specificity would be desirable; thus, further improvements in that regard will be the focus of our future studies.

Several antigens have been proposed for a potential use in the serodiagnosis of leishmaniasis, replacing crude Leishmania spp. Antigens (Maia et al., PLoS Negl Trop Dis. 2012, 6(1):e1484; Sanchez-Ovejero et al., J Proteomics. 2016, 136:145-156; Zanetti et al., Inst Med Trop Sao Paulo. 2019, 61:e42). Here we observed that serologic reactivity to NGP28b was higher in TL patients with either clinical disease or subclinical form. Subclinical L. braziliensis infection is characterized by the presence of a positive LST result in otherwise healthy subjects (Follador et al., Clin Infect Dis. 2002, 34(11):E54-8). The LST is a measure of the cellular immune response that is determined after intradermal injection of leishmanial antigens. The ensuing delayed-type hypersensitivity response is evaluated 48 hours later. A serology-based assay, such as cELISA using synthetic NGP28b, would overcome this hurdle, accelerating the diagnosis of subclinical L. braziliensis infection. A positive seroreactivity to NGP28b among subclinical individuals suggests the possibility of identifying infected (asymptomatic) individuals before the development of disease, that is, allowing for diagnosis prior to the appearance of clinical manifestations. In visceral leishmaniasis caused by L. donovani, anti-rK39 immunoassays (ELISA and dipstick tests) were used to predict disease development in contacts of VL patients (Singh et al., Clin Diag Lab Immunol. 2002, (3):568-72). By means of a prospective study, authors reported a 44% predictive value for disease development in the three months following seroevaluation, and 57% probability in six months thereafter. Given the occurrence of post-kala-azar dermal leishmaniasis (PKDL) in L. donovani infection and the probable reservoir role of asymptomatic individuals, identification of asymptomatic carriers represents an important advance in disease control. Similar advantages are expected in the case of TL caused by L. braziliensis, especially given the possibility of occurrence of severe ML or DL.

The level of IgM anti-α-Gal antibodies in CL, caused by L. mexicana or L. braziliensis, specific to the Galα1,3Man glycotope expressed on these parasite phospholipids increases with the progression of disease (Avila et al., J Clin Microbiol. 1991, (10):2305-12). Their levels are expected to be higher during active disease and decrease considerably after the decrease of the oligosaccharide stimulus provided by the parasite, suggesting that antibodies against α-Gal glycotopes could be useful for the early assessment of chemotherapeutic interventions in CL. In CL, sera obtained from individuals with active infection and post-cure recognize various α-Gal glycotopes, with different connectivity and secondary and tertiary epitopes linked to hydrophobic (lipid or protein) scaffold, on purified or synthetic molecules, indicating that numerous distinct pools of anti-α-Gal antibodies with different specificities and cross-reactivities might exist in these patients (Avila and Rojas, J Clin Microbiol. 1990, (7):1530-7)[23-25,42]. Earlier, comparisons of IgG levels to NGPs did not change drastically pre- and posttreatment in patients with CL caused by L. major (Subramaniam et al., Parasitology. 2018, 145(13):1758-1764). The inventors proposed that this was related to an accelerated recovery (lesion epithelization) time-frame (<1 month). Additionally, anti-α-Gal IgG remained high up to two years following initial detection, again in L. major-infected and cured individuals, suggesting a longevity of anti-α-Gal B-cell clones specific to L. major (Al-Salem et al., Parasitology. 2014, 141(14):1898-1903). In our setting, cured CL and ML patients still showed high IgG response to the Galα1,6Galα1,3Galfβ glycotope on NGP28b 90 days posttreatment with Sb^{v}; however, a significant decrease in anti-α-Gal levels was observed for most cured CL patients, indicating that anti-NGP28b response could be a potential BMK for the presence of active CL. In our cohort, the time-to-heal period of ML patients was significantly longer compared to CL, suggesting that circulating/residual antigens maybe maintaining the humoral response elevated, despite reepithelialization. As observed in treated adult CD patients (Pinazo et al., PLoS Negl Trop Dis. 2016, 10(1):e0004269; Torrico et al., Lancet Infect Dis. 2018, 18(4):419-430), however, a much longer treatment follow-up period would be necessary to confirm whether or not a decreasing anti-α-Gal antibody trend in CL and ML patients might correlate with the current cure criterion (reepithelialization) (Aronson et al., Clin Infect Dis. 2016, 63(12):e202-e264).

Collectively, our results show that a cELISA with a NGP based on a type-2 L. major GIPL-3 containing terminal Galα1,6Galα1,3Galfβ glycan is applicable for the serodiagnosis of TL caused by L. braziliensis, ranging from subclinical (SC) infection to severe disseminated (DL) disease, with high sensitivity and specificity. Moreover, in CL, the humoral immune response to NGP28b decreases with clinical cure indicating that this serology-based immunoassay could be potentially useful for monitoring response to chemotherapy. It will be interesting to determine, in the future, whether such Leishmania-specific anti-α-Gal antibodies persist in CL patients and, if so, whether this persistence, prospectively, may be a marker for development of mucosal disease.

B. Material and Methods

Ethics Statement. This research was conducted with the approval of the Ethical Committee of the Hospital Prof. Edgard Santos (Salvador, Bahia, Brazil; approval number 240/2009), and Comissio Nacional de Ética em Pesquisa (Brazilian National Ethics Committee, Brazil). Informed consent was obtained from each participant.

Serum samples. Sera were randomly selected from a bank of serum samples from clinically and laboratory-confirmed cases of TL identified at the Health Post of Corte de Pedra, Bahia, Brazil, a reference center for diagnosis and treatment of leishmaniasis. Epidemiological and clinical characteristics for patients with CL, ML, DL, or SC forms of TL are described in Table 3. Active TL was diagnosed by the presence of one or more ulcerative lesion(s) on the skin site(s), or in the nasal mucosa, with laboratory confirmation by detection of L. braziliensis DNA using polymerase chain reaction (PCR), or by histopathology showing amastigote forms in the tissue. Patients with DL exhibited ten or more acneiform, papular and ulcerated lesions in at least two different parts of the body. Individuals with SC infection were defined as household contacts from CL patients with a positive LST without clinical manifestations of CL. Endemic controls (EC) (n=15) consisted of household contacts of CL patients without clinical manifestations of CL, a negative LST and no production of interferon-γ in vitro. These EC individuals were not screened for any other endemic infection(s) in the region at the time of sample collection. The LST was performed with soluble leishmanial antigen, as previously described (Reed et al., Am J Trop Med Hyg. 1986, 35(1):79-85). Briefly, 25 pg of SLA was injected intradermally on the ventral face of the forearm. Test was considered positive when the induration was >5 mm after 48 h. Patients with active disease were treated daily with meglumine antimoniate—Sb^{v} (Glucantime; i.v., 20 mg/kg) for 20 days for CL and ML, and for 30 days for DL. For CL and ML patients, sera were obtained both at the time of diagnosis (day 0, active CL/ML) and following clinical confirmation of cure (day 90, cured CL/ML). For patients with other clinical forms of TL (DL and SC), sera were obtained at the time of diagnosis only. Additional sera were obtained from chronic Chagas disease (CD, n=16) patients, or from healthy nonendemic controls (NECs, n=18) residents of Salvador, BA, Brazil. NEC individuals showed negative responses to both anti-Leishmania serology and LST. The study was approved by the Institutional Review Board at the Medical School, Federal University of Bahia.

Neoglycoproteins (NGPs). Mercaptopropyl glycoside derivatives were synthetized and then chemically coupled to bovine serum albumin (BSA), used as carrier protein, to generate NGP30b (Galpα1,3Galfβ1,3Manα-BSA) and NGP28b (Galp1,6Galpα1,3Galfβ-BSA), which were based on L. major type-2 GIPL-2 and -3, respectively, as described (Montoya et al., JACS Au. 2021, 1(8):1275-1287). NGP29b (Galfβ1,3Manα-BSA), based on L. major type-2 GIPL-1, was synthesized following the same methodology as recently described (Montoya et al., Molecules. 2022, 27(2):411).

Chemiluminescent enzyme-linked immunosorbent assay (cELISA). To determine levels of anti-α-Gal IgG antibodies, the NGPs were cross-titrated at concentrations ranging from 3.13 to 50 ng/well using sera pooled from NEC (n=15) individuals, or CL (n=15), ML (n=15), or CD (n=15) patients, at 1:400 or 1:800 dilution. Initially, white opaque 96-well MaxiSorp Immune Plates (catalog number 436110, Thermo Fisher Scientific) were coated with NGPs overnight (0/N) at 4° C. in 100 mM carbonate-bicarbonate buffer, pH 9.6 (CBB). Wells were blocked with 200 μL PBS-1% BSA (PBS-B) for 1 h, at 37° C. Human serum samples, diluted in PBS-B plus 0.05% Tween 20 (PBS-TB) were then added and incubated for 1 h, at 37° C. After washing, plates were sequentially incubated with 50 μL biotinylated goat antihuman IgG (H+L) secondary antibody (1:5,000 dilution in PBS-TB; catalog number 31030, Thermo Fisher Scientific), and 50 μL Pierce High Sensitivity NeutrAvidin-horseradish peroxidase (1:5,000 dilution in PBS-TB; catalog number 31030, Thermo Fisher Scientific). Incubation steps were performed for 30 min at 37° C. Between incubation steps, plates were washed 3× with 250 μL PBS-T. The reaction was developed with SuperSignal ELISA Pico Chemiluminescent Substrate (catalog number 37069, Thermo Fisher Scientific) by diluting the Luminol/Enhancer Solution and Stable Peroxide Solution in CBB, at 1/1/8 ratio (v/v/v). Luminescence was read in a FilterMax F3 Microplate Reader (Molecular Devices) and values expressed as relative luminescent units (RLUs). Pools of sera from 15 active CL (aCL), cured CL (cCL) individuals, and 15 NECs were also used as positive (aCL) and negative (cCL and NECs) controls. Serum sample from each patient was tested in technical triplicate. The mean (K) RLU value was normalized (as cELISA titer) by dividing it by the cutoff value, calculated as follows: cutoff=x̅+SDf, where x̅ is the mean value of six technical replicates of a pool of sera from NECs in each microplate; SDf is the standard deviation (SD) multiplier, calculated based on the number of negative control replicates in each microplate, as described (Frey et al., J Immunol Methods. 1998, 221(1-2):35-41). The titer of each cELISA was defined as the ratio of the experimental sample's average RLU value to the cutoff value. A serum sample was considered positive when its cELISA titer was equal to or higher than 1.000, and negative when the titer was lower than 1.000, as previously described (Montoya et al., JACS Au. 2021, 1(8):1275-1287).

Statistical Analysis. The variables in this study were evaluated regarding their distribution with the Kolmogorov-Smirnov test and skewness and kurtosis values obtained using IBM SPSS Statistics 20 software. Once data showed a nonparametric distribution, analyses were performed using Kruskal-Wallis followed by Dunn's post-test. Cross-titration curves were compared using two-way Anova with main effects only and Dunnett's multiple comparison test (with individual variances computed for each comparison). Paired comparisons (pre- and posttreatment) were performed using the Wilcoxon Rank Sum test. Statistical significance was set at the conventional 5% level (p<0.05) and all non-parametric analyses were performed using GraphPad Prism version 9.0 (GraphPad Software, San Diego, Calif.). Finally, multiple logistic regression models followed by receiver-operating characteristic (ROC) curve analyses were performed on normalized (cELISA titer) values to establish sensitivity, specificity, and other performance parameters obtained from ROC/AUC, two-graph ROC (TG-ROC), p values, and likelihood ratio, using GraphPad Prism v. 9.0.

1. A neoglycoconjugate comprising a glycan coupled to a carrier, wherein the glycan comprises Galpα1,3Galfβ; Galpα1,6Galpα1,3Galfβ; or Galpα1,3Galfβ1,3Manpα.

2. The neoglycoconjugate of claim 1 wherein the carrier is a protein carrier.

3. The neoglycoconjugate of claim 2, wherein the protein carrier is bovine serum albumin.

4. The neoglycoconjugate of claim 1, comprising 5 to 50 glycans per protein carrier.

5. The neoglycoconjugate of claim 1, further comprising a linker connecting the glycoside to the carrier.

6. The neoglycoconjugate of claim 5, wherein the linker comprises —(CH₂)ₓS—, wherein x is an integer between 1 to 10.

7. The neoglycoconjugate of claim 6, wherein x is 3.

8. A method of detecting a parasite, the method comprising:
contacting a blood sample from a subject with the neoglycoconjugate of claim 1; and
detecting binding between the neoglycoconjugate with antibodies in the blood sample that bind a glycan having a terminal αGal.

9. The method of claim 8, wherein the binding is detected using enzyme-linked immunosorbent assay (ELISA).

10. The method of claim 8, wherein the subject is a human.

11. The method of claim 8, wherein the subject is suspected of having cutaneous leishmaniasis (CL).

12. The method of claim 8, wherein the parasite is Leishmania major.

13. A method of diagnosing cutaneous leishmaniasis in a subject, the method comprising:
contacting a blood sample from a subject with the neoglycoconjugate of claim 1;
detecting whether the neoglycoconjugate binds with antibodies in the blood sample that bind a glycan having a terminal, nonreducing α-Gal; and
diagnose the subject with cutaneous leishmaniasis if binding between the neoglycoconjugate and the antibodies is detected.

14. The method of claim 13, wherein the subject is a human.

15. A method of treating cutaneous leishmaniasis in a subject, the method comprising:
contacting a blood sample from a subject with the neoglycoconjugate claim 1;
detecting whether the neoglycoconjugate binds with antibodies in the blood sample that bind a glycan having a terminal, nonreducing α-Gal; and
administering a treatment of cutaneous leishmaniasis if binding between the neoglycoconjugate and the antibodies is detected.

16. The method of claim 15, wherein the subject is a human.

17. (canceled)

18. A glycoside comprising a glycan selected from Galpα1,3Galfβ, Galpα1,6Galpα1,3Galfβ, or Galpα1,3Galfβ1,3Manpα.

19. The glycoside of claim 18, having the chemical formula of Galpα1,3Galfβ(CH₂)ₓSH, Galpα1,6Galpα1,3Galfβ(CH₂)ₓSH, or Galpα1,3Galfβ1,3Manpα(CH₂)ₓSH, wherein x is, independently, an integer from 1 to 10.

20. The glycoside of claim 19, wherein x is 3.
